(19) United States
(12) Patent Application Publication
(10) Pub. No.: US 2023/0091430 A1
(43) Pub. Date: Mar. 23, 2023

(54) TREATMENT OF ATOPIC DERMATITIS EMPLOYING ANTI-IL-13Ra1 ANTIBODY OR BINDING FRAGMENT THEREOF IN AN ALLERGIC POPULATION

(71) Applicant: Aslan Pharmaceuticals PTE LTD, Singapore (SG)

(72) Inventors: Karen VEVERKA, Singapore (SG); Alison WARD, Singapore (SG)

(21) Appl. No.: 17/929,874

(22) Filed: Sep. 6, 2022

Related U.S. Application Data
(63) Continuation-in-part of application No. PCT/SG2022/050103, filed on Mar. 1, 2022.

(30) Foreign Application Priority Data
Mar. 1, 2021 (SG) 10202102087T
Sep. 27, 2021 (SG) 10202110690S

(51) Int. Cl. C07K 16/28; A61P 17/00

(52) U.S. Cl. CPC C07K 16/2866; A61P 17/00; A61K 2039/505

(57) ABSTRACT

An antibody, antigen binding fragment thereof and compositions comprising the same, which is an inhibitor of signalling through IL-13Rα1, by binding the said receptor, for use in the treatment of allergic disease, such as atopic dermatitis (for example moderate to severe atopic dermatitis, in particular poorly controlled moderate to severe atopic dermatitis) in a highly allergic patient where the baseline IgE levels have been established and are at a level of at least 10,000 KU/L+/−2,000.

### CROSS-REFERENCE TO RELATED APPLICATIONS

This application is a Continuation-in-Part of International Patent Application No. PCT/SG2022/050103 filed on Mar. 1, 2022, which claims priority to Singapore Application No. 10202110690S filed on Sep. 27, 2021 and Singapore Application No. 10202102087T filed on Mar. 1, 2021, the content of each of which applications is incorporated herein by reference.

### INCORPORATION OF SEQUENCE LISTING

This application contains a sequence listing submitted electronically via EFS-web, which serves as both the paper copy and the computer readable form (CRF) and consists of a file entitled “STAPL17CIP_seqlist.xml”, which was created on Sep. 6, 2022, which is 83,245 bytes in size, and which is herein incorporated by reference in its entirety.

The present disclosure relates to use of an anti-IL-13Rα1 antibody or binding fragment thereof or a pharmaceutical composition comprising same to treat atopic dermatitis in a highly allergic population.

### BACKGROUND

A way to inhibit the activity of IL-13 is to interfere with the binding of IL-13 to its receptor IL-13R, for example by using an antibody specific to IL-13R, such as an antibody specific to IL-13Rα1. An effective antibody antagonist to IL-13Rα1 may also interfere with the binding of IL-13 and prevent heterodimerization of IL-4Rα and IL-13Rα1. Such an antibody could inhibit signaling of both IL-13 and IL-4 through the type II receptor while sparing IL-4 signalling through the type I receptor. Signalling through the type I receptor is essential in the induction phase of the immune response during which Th2 cells differentiate. T cells do not express IL-13Rα1 so the type II receptor plays no role in Th2 differentiation. Hence, an IL-13Rα1 antibody should not affect the overall Th1/Th2 balance. Signalling through the type II IL-4/IL-13 receptor is critical during the effector-A-stage of the immune response during established allergic inflammation. Thus, blockade of the type II receptor should have a beneficial effect on many of the symptoms of conditions mediated by IL-13R-mediated and therefore, be an effective disease modifying age.

Antibodies against IL-13Rα1 (both monoclonal and polyclonal) have been described in the art; see, eg, WO97/15663, WO03/80675; WO03/46009; WO06/072564; Gauchat et al, 1998 Eur. J. Immunol. 28:4286-4298; Gauchat et al, 2000 Eur. J. Immunol. 30:3157-3164; Clement et al, 1997 Cytokine 9(11):959 (Meeting Abstract); Ogata et al, 1998 J. Biol. Chem. 273:9864-9871; Graber et al, 1998 Eur. J. Immunol. 28:4286-4298; C. Vermot-Desroches et al, 2000 Tissue Antigens 5 (Supp. 1):52-53 (Meeting Abstract); Poudrier et al, 2000 Eur. J. Immunol. 30:3157-3164; Akaiwa et al, 2001 Cytokine 13:75-84; Cancino-Diaz et al, 2002 J. Invest Dermatol. 119:1114-1120; and Krause et al, 2006 MoI. Immunol. 43:1799-1807.

One particularly promising anti-IL-13Rα1 antibody is described in WO2008/060813 as antibody 10G5-6. 10G5-6 as an IgG4 with a hinge stabilising serine to proline mutation (S241P Kabat numbering) and is known as ASLAN004. ASLAN004 has been shown to bind to human IL-13Rα1 with a high affinity (for example Kd may be 500 μM). ASLAN004 was shown to effectively antagonise IL-13 function through inhibiting the binding of IL-13 to its receptor IL-13Rα1 and to inhibit IL-13 and IL-4 induced eotaxin release in NHDF cells, IL-13 and IL-4 induced STAT6 phosphorylation in NHDF cells and IL-13 stimulated release of TARC in blood or peripheral blood mononuclear cells.

Atopic dermatitis (AD) can be a very painful, demoralising and psychologically damaging disease. There are a number of underlying causes of the disease and markers such as TARC are employed to track the disease prognosis, and status.

IgE levels may also be assessed and data is starting to suggest that different categories of patients exist within broader group of atopic dermatitis.

Dupilumab at phase 2 the patient population had a baseline IgE value in the region of 7,000 KU/L.

The present inventors have treated patients with a baseline IgE level in the region of 14,000 KU/L. This represents a potentially more allergic patient population.

The present inventors have established that these patients with higher baseline levels of IgE can be treated and respond well to treatment with an anti-IL-13Rα1 antibody or binding fragment thereof. This is surprising because these patients have higher levels of inflammation and hence are likely to me more difficult to treat, for example may be more difficult to get results where the disease is modified (for example as measure by EASI score) and/or may take longer to obtain results. What is more the positive response may be achieved in the target population within only 8 weeks (i.e. about day 57). The percentage reduction from baseline (albeit the same numerical percentage reduction) is in fact a much a larger reduction in real terms for patients with higher starting levels of IgE.

It may be particularly beneficial to target this population of patients for treatment.

### SUMMARY OF THE DISCLOSURE

The present disclosure will be summarised by the following paragraphs:
1. An antibody or antigen binding fragment thereof, which is an inhibitor of signalling through IL-13Rα1, by binding the said receptor, for use in the treatment of allergy such as atopic dermatitis (for example moderate to severe atopic dermatitis, in particular poorly controlled moderate to severe atopic dermatitis) in a highly allergic patient, for example where the baseline IgE levels have been established and are at a level of at least 10,000 KU/L+/−2,000.
2. An antibody or antigen binding fragment thereof for use according to paragraph 1 wherein the baseline IgE levels are in the range 10,000+/−2,000 to 30,000+/−6,000 KU/L.
3. An antibody or antigen binding fragment thereof for use according to paragraph 1 or 2, wherein the treatment reduces the IgE levels by at least 15% from baseline.
4. An antibody or antigen binding fragment thereof for use according to paragraph 3, wherein the treatment reduces the IgE levels by at least 20% from the baseline.
5. An antibody or antigen binding fragment thereof for use according to paragraph 3 or 4, wherein the treatment reduces the IgE levels by at least 30% from baseline, for example reduces said levels 30 to 40%, such as 30, 31, 32, 33, 34, 35, 36, 37, 38, 39 or 40% from baseline.
6. An antibody or antigen binding fragment according to any one of paragraphs 3 to 5, wherein the reduction is observed by about day 15.
7. An antibody or antigen binding fragment thereof according to any one of paragraphs 3 to 6, wherein the reduction is observed by about day 29.
8. An antibody or antigen binding fragment thereof according to any one of paragraphs 3 to 7, wherein the reduction is observed by about day 57.
9. An antibody or antigen binding fragment thereof for use according to any one of paragraphs 1 to 8, wherein said antibody or binding fragment is administered parenterally, for example in a treatment cycle comprising a dose in the range 200 mg to 600 mg.
10. An antibody or antigen binding fragment thereof for use according to any one of paragraphs 1 to 9, wherein the treatment is administered intravenously.
11. An antibody or antigen binding fragment thereof for use according to any one of paragraphs 1 to 9, wherein the treatment is administered subcutaneously.
12. An antibody or antigen binding fragment thereof for use according to any one of paragraphs 1 to 11, wherein multiple doses are administered in a treatment cycle (for example wherein the treatment cycle is 4 to 8 weeks, such as 8 weeks).
13. An antibody or binding fragment thereof for use according to paragraph 12, wherein multiple treatment cycles are administered, for example 2, 3, 4 or more treatment cycles are administered.
14. An antibody or binding fragment thereof for use according to paragraph 12 or 13, wherein following the treatment cycle or cycles and disease modification, maintenance therapy is administered, for example the same dose administered less frequently (for example monthly), or a lower dose (such as 200 mg) administered the same frequency or less frequently (such as about two weekly, about three weekly, or about four weekly).
15. An antibody or antigen binding fragment for use according to any one of paragraphs 1 to 14, wherein said antibody or binding fragment thereof is administered approximately weekly, (in particular a single treatment cycle, especially 8 weeks).
16. An antibody or antigen binding fragment for use according to any one of paragraphs 1 to 14, wherein said antibody or binding fragment thereof is administered once approximately every two weeks, (in particular a single treatment cycle, especially 8 weeks).
17. An antibody or antigen binding fragment for use according to any one of paragraphs 1 to 14, wherein said antibody or binding fragment thereof is administered once approximately every three weeks, (in particular a single treatment cycle, especially 8 weeks).
18. An antibody or antigen binding fragment for use according to any one of paragraphs 1 to 14, wherein the antibody or binding fragment thereof is administered once approximately every four weeks (for example monthly), (in particular a single treatment cycle, especially 8 weeks).
19. An antibody or antigen binding fragment for use according to any one of paragraphs 1 to 18, wherein a loading dose in the range 400 to 900 mg, for example 400, 500, 600, 700, 800 or 900 mg is employed before administration of the treatment cycle.
20. An antibody or antigen binding fragment for use according to any one of paragraphs 1 to 18, wherein the treatment does not comprise a loading dose.
21. An antibody or antigen binding fragment for use thereof according to any one of paragraphs 1 to 20, wherein the dose is 200 mg.
22. An antibody or binding fragment thereof for use according to any one of paragraphs 1 to 20, wherein the dose is in the range 350 to 450 mg, such as 400 mg.
23. An antibody or binding fragment thereof for use according to any one of paragraphs 1 to 20, wherein the dose is 600 mg.
24. An antibody or binding fragment for use according to any one paragraphs 1 to 23, wherein the treatment cycles comprises, a first dose at 600 mg, followed by three weekly doses of 400 mg, for example wherein the treatment cycle is repeated twice i.e. two treatment cycles lasting 8 weeks, in particular day 1 600 mg, approximately day 8 400 mg, approximately day 15 400 mg, approximately day 22 400 mg, approximately day 29 600 mg, approximately day 36 400 mg, approximately day 43 400 mg, and approximately day 50 400 mg are administered.
25. An antigen binding fragment for use according to any one of paragraphs 1 to 24, wherein disease modification, occurs by day 4, wherein day 1 is the first administration of the antibody or binding fragment thereof, for example wherein disease modification is assessed by a clinically relevant score, for example reduction in EASI score or IGA, in particular EASI score.
26. An antibody or antigen binding fragment thereof for use according to any one of paragraphs 1 to 25, wherein the antibody or binding fragment binds an epitope FFYQ (for example same epitope as the antibody with a VH shown in SEQ ID NO: 51 and a VL shown in SEQ ID NO: 53, in particular an antibody or binding fragment with a sequence disclosed herein.
27. An antibody or antigen binding fragment thereof for use according to any one of paragraphs 1 to 26, wherein the anti-IL-13R antibody comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 10.
28. An antibody or antigen binding fragment thereof for use according to any one of paragraphs 1 to 27, wherein the anti-IL-13R antibody comprises a VH domain comprising an amino acid sequence shown in SEQ ID NO: 51 or a sequence at least 95% identical thereto.
29. An antibody or antigen binding fragment thereof for use according to any one of paragraphs 1 to 28, wherein the anti-IL-13R antibody comprises a VL CDR1 comprising an amino acid sequence as set forth in SEQ ID NO: 31, a VL CDR2 comprising an amino acid sequence as set forth in SEQ ID NO: 32, and a VL CDR3 comprising an amino acid sequence as set forth in SEQ ID NO: 45.
30. An antibody or antigen binding fragment thereof for use according to any one of paragraphs 1 to 29, wherein the anti-IL-13R antibody comprises a VL domain comprising an amino acid sequence shown in SEQ ID NO: 53 or a sequence at least 95% identical thereto.
31. A pharmaceutical formulation comprising an antibody or binding fragment according to any one of paragraphs 1 to 30, said formulation comprising: 10 to 140 mg/ml of the antibody or binding fragment (for example 10, 15, 20, 25, 30, 35, 40, 45, 50, 55, 60, 65, 70, 75, 80, 85, 90, 95, 100, 105, 110, 115, 120, 125, 130, 135, 140 mg/ml); 50 mM to 150 mM of arginine (for example 50, 55, 60, 65, 70, 75, 80, 85, 90, 95, 100, 105, 110, 115, 120, 125, 130, 135, 140, 145 or 150, such as 100 mM arginine); 15 to 25 mM histidine buffer, for example 15, 16, 17, 18, 19, 20, 21, 22, 23, 24 and 25, such as 20 mM histidine buffer; 0.01-0.03% of a non-ionic surfactant, such as 0.02% w/v and the pH of the formulation is in the range 5.5 to 7.5 for example 6.2 to 7.2 (such as 6.2, 6.3, 6.4, 6.5, 6.6, 6.7, 6.8, 6.9, 7.0, 7.1, 7.2), such as 6.5 to 7.0, in particular 6.4 to 6.9), for use in the treatment of atopic dermatitis (for example moderate to severe atopic dermatitis, in particular poorly controlled moderate to severe atopic dermatitis) in a highly allergic patient where the baseline IgE levels have been established and are at a level of at least 10,000 KU/L+/−2,000 (in particular for use as defined in any one of claims 1 to 27).
32. A pharmaceutical formulation for use according to paragraph 31 wherein said formulation comprises: 10 to 140 mg/ml of the antibody or binding fragment; 50 mM to 150 mM of arginine (for example 50, 55, 60, 65, 70, 75, 80, 85, 90, 95, 100, 105, 110, 115, 120, 125, 130, 135, 140, 145 or 150, such as 100 mM arginine); 15 to 25 mM histidine buffer, for example 15, 16, 17, 18, 19, 20, 21, 22, 23, 24 and 25, such as 20 mM histidine buffer; 0.01-0.03% of a non-ionic surfactant, such as 0.02% w/v and wherein the pH of the formulation is in the range 5.5 to 7.5 for example 6.2 to 7.2 (such as 6.2, 6.3, 6.4, 6.5, 6.6, 6.7, 6.8, 6.9, 7.0, 7.1, 7.2), such as 6.5 to 7.0, in particular 6.4 to 6.9)
33. A pharmaceutical formulation for use according to paragraph 31 or 32, wherein the osmolarity of the formulation is in the range 350 to 550 mOsmo/kg, for example 350, 355, 365, 370, 375, 380, 385, 390, 395, 400, 405, 410, 415, 420, 425, 430, 435, 440, 445, 450, 455, 460, 465, 470, 475, 480, 485, 490, 495, 500, 505, 515, 520, 525, 530, 535, 540, 545, 550, such as 405 to 435 mOsmo/kg.
34. A pharmaceutical formulation for use according to any one of paragraphs 31 to 30, which further comprises 50 to 200 mM of a sugar, for example 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, such as 180 mM sugar.
35. A pharmaceutical formulation for use according to any one of paragraphs 31 to 34, wherein the pH is 6.5.
36. A pharmaceutical formulation for use according to any one of paragraphs 31 to 35, wherein the formulation does not comprise NaCl.
37. A pharmaceutical formulation for use according to any one of paragraphs 31 to 35, wherein the formulation comprises 50 to 150 mM of NaCl, for example 50, 55, 60, 65, 70, 75, 80, 85, 90, 95, 100, 105, 110, 115, 120, 125, 130, 135, 140, 145, 150, such as 62.5 or 140 mM NaCl.

In one embodiment there is provided a method of treating a highly allergic patient as defined herein with an antibody or binding fragment thereof or a pharmaceutical formulation as defined herein.

In one embodiment there is provided use of an antibody or binding fragment thereof or a pharmaceutical formulation, as defined herein for the manufacture of a medicament for the atopic dermatitis in a highly allergic patient.

In one embodiment there is a provided a reduction in the Investigator Global Assessment (IGA) with/after treatment according to the present disclosure, for example an assessment of 0, 1 or 2, (no inflammatory signs, almost clear and mild disease respectively). In particular there is provided an IGA score of 0 or 1.

In one embodiment a combination therapy is employed comprising the antibody, antigen binding fragment thereof or a formulation according to the present disclosure and a further medicament. In one embodiment the further medicament is for the treatment of atopic dermatitis, for example topical steroids, oral steroids, and/or antihistamines.

Surprisingly disease modification following treatment with an anti-IL-13Rα1 antibody or binding fragment thereof according to the present disclosure closely follows reduction in TARC (thymus-and-activation regulated chemokine, also known as CCL17), in fact the TARC reduction and EASI reduction correlate closely.

Hence, in one embodiment, there is provided a reduction in TARC levels with/after treatment according to the present disclosure, for example a % change from baseline TARC levels of −15% to −100%, such as −15%, −20%, −25%, −30%, −35%, −40%, −45%, −50%, −55%, −60%, −65%, −70%, −75%, −80%, −85%, −90%, −95% or −100%. In one embodiment, the % change from baseline TARC levels is in the range of −20% to −100%, such as −20%, −25%, −30%, −35%, −40%, −45%, −50%, −55%, −60%, −65%, −70%, −75%, −80%, −85%, −90%, −95% or −100%.

In one embodiment, there is % change from baseline TARC levels of at least 50%, such as −50%, −55%, −60%, −65%, −70%, −75%, −80%, −85%, −90%, −95% or −100%. In one embodiment, the % change from baseline TARC level is in the range of −60 to −80%, for example −60%, −65%, −70%, −75% or −80%.

In one embodiment, the treatment reduces the IgE levels by at least 15% from baseline, such as −15%, −20%, −30%, −40%, −50%, −60%, −70%, −80%, −90%, −100%. In one embodiment, the treatment reduces the IgE levels by at least 20% from the baseline, for example −20%, −30%, −40%, −50%, −60%, −70%, −80%, −90%, −100. In one embodiment the treatment reduces the IgE levels by at least 30% from baseline, for example reduces said levels 30 to 40%, such as 30, 31, 32, 33, 34, 35, 36, 37, 38, 39 or 40% from baseline.

In one embodiment, there is further provided a reduction in lactate dehydrogenase (LDH) levels with/after treatment according to the present disclosure, for example a % change from baseline LDH levels of −15% to −100%, such as −15%, −20%, −25%, −30%, −35%, −40%, −45%, −50%, −55%, −60%, −65%, −70%, −75%, −80%, −85%, −90%, −95% or −100%. In one embodiment, the % change from baseline LDH levels is at least 20% such as −20%, −30%, −35%, −40%, −45%, −50%, −55%, −60%, −65%, −70%, −75%, −80%, −85%, −90%, −95% or −100%. In one embodiment, the % change from baseline LDH is in the range of −15% to −35%, for example −15%, −20%, −25%, −30% or −35%.

Surprisingly disease modification following treatment with an anti-IL-13Rα1 antibody or binding fragment thereof according to the present disclosure results in a reduction in LDH levels, in fact the LDH reduction, IgE and/or TARC reduction correlate closely.

The present inventors have also established that the antibodies, antigen binding fragments and compositions of present invention can be employed to treated other diseases with an allergic component, for example allergic epithelial disease, such as allergic asthma, asthma-COPD and eosinophilic esophagitis.

Thus, in an independent aspect there is provided treatment of an allergic disease, for example with elevated IgE levels (elevated in comparison to normal levels), with an antibody, binding fragment or composition comprising the same, which is an inhibitor of signalling through IL-13Rα1, by binding the said receptor, for example as described elsewhere herein (for atopic dermatitis).

Whilst not wishing to be bound by theory, the present inventors believe that the same cytokines and similar or corresponding mechanisms are at work in these allergic diseases as in this population of atopic dermatitis.

In one embodiment the allergic disease is manifested in epithelial tissue.

In one embodiment the allergic disease is allergic asthma, for example poorly controlled and/or moderate to severe asthma.

In one embodiment the allergic disease is asthma-COPD, for example poorly controlled and/or moderate to severe asthma-COPD.

In one embodiment the allergic disease is eosinophilic esophagitis, for example poorly controlled and/or moderate to severe eosinophilic esophagitis.

In one embodiment the patient is identified as allergic before treatment, for example where the baseline has been established and is at a level of at least 10,000 KU/L+/−2,000.

Preferences described herein for atopic dermatitis, like dose and the like apply equally to allergic disease indications.

In one embodiment the allergic disease is not atopic dermatitis.

In one embodiment the allergic disease is not eosinophilic esophagitis.

### DETAILED DISCLOSURE

Disease modification as employed herein relates to improvements in the disease status for example as measured by a clinically relevant score, in particular a reduction in the EASI score.

A clinically relevant score is a score used in the clinical, for example used by a physician.

In one embodiment the disease is modified by a percentage reduction in Eczema Area and Severity Index (EASI) score in the range −20 to −100% from the baseline, such as EASI 50, EASI 75 or EASI 90. EASI score and EASI are used interchangeably herein.

Eczema Area and Severity Index (EASI) score as used herein is a tool used to measure the area (which indicates the extent of disease) and severity of atopic eczema. The number after the term “EASI” indicates the % decrease in the score from baseline. Thus, EASI 50 for example refers to a 50% decrease in the score and EASI 90 refers to a 90% decrease in the score.

In one embodiment disease modification is measured as a reduction in IGA.

Investigator's global assessment (IGA) as used herein refers to a tool for the assessment of atopic dermatitis. It uses a 0-5 point scale depending on the severity of a patient's symptoms:

| Score | Severity | Description of symptoms |
|---|---|---|
| 0 | clear | no inflammatory signs |
| 1 | almost clear | Just perceptible erythema and just perceptible |
| | | papulation/infiltration |
| 2 | mild disease | Mild erythema and mild papulation/infiltration |
| 3 | moderate disease | moderate erythema and moderate papulation/infiltration |
| 4 | severe disease | severe erythema and severe papulation/infiltration |
| 5 | very severe disease | severe erythema and severe papulation/infiltration with |
| | | oozing/crusting |

Patient Oriented Eczema Measure (POEM) as used herein refers to a 7-item questionnaire for patients that assesses presence of disease symptoms (dryness, itching, flaking, cracking, sleep loss, bleeding, and weeping) over the last week using a scoring system of 0 (no days) to 4 (every day). The total score ranges from 0 to 28 with higher scores indicating greater intensity of eczema.

In an independent aspect there is provided use of an antibody, antigen binding fragment or pharmaceutical formulation as disclosed herein for the treatment or amelioration of allergic food responses, in particular severe allergic food response, such as peanut allergy.

In one embodiment, the highly allergic patient's baseline IgE levels have been established and are at a level of at least at least 10,000 KU/L+/−2,000, such as 8000, 8500, 9000, 9500, 10000, 10500, 11000, 11500 or 12000 KU/L.

In one embodiment, the patient has been identified as a highly allergic patient before the treatment is administered, for example, wherein the patient's baseline IgE levels have been established and are at a level of at least 10,000 KU/L+/−2,000. Thus, in one embodiment the patient is identified as a highly allergic patient wherein the patient's baseline IgE levels have been established and are at a level of at least 10,000 KU/L+/−2,000, prior to administration of the antibody or binding fragment thereof, pharmaceutical formulation or medicament as defined herein. Accordingly, in one embodiment the target population to be treated is highly allergic patients whose baseline IgE levels have been established and are at a level of at least 10,000 KU/L+/−2,000.

In one embodiment the patient is identified as highly allergic by clinical observation.

Asthma is a respiratory disease characterized by inflammation and bronchospasm, wherein the muscles around the airways tighten and contract in an attempt to keep the airways open. This leaves patients with cough, wheezing, chest tightness and shortness of breath. When the breathing issues become severe, this is typically referred to as an asthma attack.

Allergic asthma (used interchangeably with allergy-induced asthma) as used herein refers to form of asthma whereby the lungs of a patient become inflamed, and the airways tighten in response to the inhalation of an allergen. Common allergens include pollen, dust, animal dander and mold. Patients with allergic asthma experience many of the same symptoms as patients with non-allergic asthma—cough, wheezing, chest tightness and shortness of breath. Hence, the major difference between the two conditions is that patients with allergic asthma normally experience symptoms after inhaling an allergen.

Chronic obstructive pulmonary disease (COPD) is a collection of chronic inflammatory lung diseases that obstruct airflow and result in breathing problems. Emphysema and chronic bronchitis are the two most common conditions that contribute to COPD. Empysema is a condition wherein the alveoli are damaged and chronic bronchitis is a condition whereby the bronchial tubes are inflamed. These two conditions usually occur together and can vary in severity among individuals with COPD.

Asthma-COPD (used interchangeably with asthma-COPD overlap syndrome (ACOS)) as used herein refers to a condition whereby a patient has both asthma and COPD. Patients diagnosed with asthma-COPD have reduced lung function, have more severe asthma attacks and typically experience symptoms more frequently than patients with asthma or COPD alone. Hence, asthma-COPD is a more serious and dangerous condition than either disease alone.

Eosinophilic esophagitis (EOE) as used herein refers to a chronic immune system disease where eosinophils build up in the esophagus (eosinophils are not normally found in the esophagus). This build-up occurs as a reaction to foods, allergens or acid reflux, and may inflame and cause injury to the esophageal tissue. This in turn may lead to narrowing of the esophagus and difficulty swallowing. In serious cases it may even result in medical emergencies due to food getting stuck in the throat. The majority of patients with EOE are atopic. Thus, individuals with atopic dermatitis, food or other environmental allergies are at greater risk of developing EOE. Family history of EOE is also a risk factor for the condition. No medications are currently approved by the US FDA for treating EOE. However, proton pump inhibitors (PPIs) have been demonstrated to reduce esophageal inflammation in some EOE patients and are thus often used as a first treatment. Corticosteroids may also be administered to help control inflammation.

Interleukin-13 receptor (IL-13R) as used herein is a type I cytokine receptor, which binds to Interleukin-13. It consists of two subunits, encoded by IL13Rα1 and IL4R, respectively. These two genes encode the proteins IL-13Rα1 and IL-4Rα. These form a dimer with IL-13 binding to the IL-13Rα1 chain and IL-4Rα stabilises this interaction. Due to the presence of the IL4R subunit, IL13R can also instigate IL-4 signalling. In both cases this occurs via activation of the Janus kinase (JAK)/Signal Transducer and Activator of Transcription (STAT) pathway, resulting in phosphorylation of STATE. Human IL-13Rα1 has the Uniprot number P3597.

IL-13Rα2, previously called IL-13R and IL-13Rα, is another receptor which is able to bind to IL-13. However, in contrast to IL-13Rα1, this protein binds IL-13 with high affinity, but it does not bind IL-4. Human IL-13Rα2 has the Uniprot number Q14627.

In one embodiment the anti-IL-13R antibody or binding fragment thereof of the present disclosure binds to IL-13Rα1. In one embodiment, the antibody or binding fragment thereof binds only to IL-13Rα1 and does not bind to IL-13Rα2.

In one embodiment CDRH1 comprises an amino acid sequence GYSFTSYWIG (SEQ ID NO: 1) as disclosed in WO2020/197502 incorporated herein by reference.

In one embodiment CDRH2 comprises an amino acid sequence VIYPGDSYTR (SEQ ID NO: 2) as disclosed in WO2020/197502 incorporated herein by reference.

In one embodiment CDRH3 comprises the formula:

SEQ ID NO: 3
X₁ Pro Asn Trp Gly X₆ X₇ Asp X₉

X₁ denotes Phe, Met, Gln, Leu or Val
X₆ denotes Ser or Ala
X₇ denotes Phe, Leu, Ala or Met
X₉ denotes Tyr, Gln, Lys, Arg Trp, His, Ala, Thr, Ser, Asn or Gly

In one embodiment the IL13-R1a1 antibody or binding fragment employed in the formulation of the present disclosure comprises a CDRH3 independently selected from a sequence comprising SEQ ID NO: 4 to 30.

In one embodiment, the anti-IL13R antibody or binding fragment employed in the present disclosure comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: or 3.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4, 5, 6, 7, 8, 9, 10, 11, 12, 13, 14, 15, 16, 17, 18, 19, 20, 21, 22, 23, 24, 25, 26, 27, 28, 29 or 30.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10.

In one embodiment CDRL1 is an amino acid sequence comprising RASQSISSSYLA SEQ ID NO: 31.

In one embodiment CDRL2 is an amino acid sequence comprising GASSRAT SEQ ID NO: 32

In one embodiment CDL3 comprises the formula:

SEQ ID NO: 33
Gln X₂X₃X₄X₅

X₂ denotes Gln, Arg, Met, Ser, Thr or Val.
X₃ denotes Tyr or Val.
X₄ denotes Glu, Ala, Gly or Ser.
X₅ denotes Thr, Ala or Ser.

In one embodiment the IL-13Rα1 antibody employed in the formulation of the present disclosure comprises a CDRL3 independently selected from a sequence comprising SEQ ID NO: 34 to 47.

In one embodiment, the anti-IL-13Rα antibody or binding fragment employed in the present disclosure comprises a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 33.

In one embodiment, the anti-IL-13Rα antibody of the present disclosure comprises a VL CDR1 comprising an amino acid sequence SEQ ID NO: 31, a VL CDR2 comprising an amino acid sequence SEQ ID NO: 32, and a VL CDR3 comprising an amino acid sequence as set forth in SEQ ID NO: 34 35, 36, 37, 38, 39, 40, 41, 42, 43, 44, 45, 46, or 47.

In one embodiment, the anti-IL-13Rα antibody of the present disclosure comprises a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: or 3, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 33.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3 or 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 34, 35, 36, 37, 38, 39, 40, 41, 42, 43, 44, 45, 46, or 47.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3 or 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment the VH region is independently selected from a sequence comprising the group comprising: SEQ ID NO: 48, SEQ ID NO: 49, SEQ ID NO: 50 and SEQ ID NO: 51, or a sequence at least 95% identical to any one of the same.

SEQ ID NO: 48
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
FPNWGSFDYWGQGTLVTVSS
SEQ ID NO: 49
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCA
RMPNWGSFDYWGQGTLVTVSS
SEQ ID NO: 50
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCVR
MPNWGSLDHWGQGTLVTVSS
SEQ ID NO: 51
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSLDHWGQGTLVTVSS
In one embodiment the VL is independently selected
from the group comprising SEQ ID NO: 52, SEQ ID
NO: 53 and SEQ ID NO: 54, or a sequence at least
95% identical to any one of the same.
SEQ ID NO: 52
EIVLTQSPGTLSLSPGERATLSCRASQSISSSYLAWYQQKPGQAPRLLI
YGASSRATGIPDRFSGSGSGTDFTLTISRLEPEDFAVYYCQQYETFGQG
TKVEI*
SEQ ID NO: 53
EIVLTQSPGTLSLSPGERATLSCRASQSISSSYLAWYQQKPGQAPRLLI
YGASSRATGIPDRFSGSGSGTDFTLTISRLEPEDFAVYYCQQYASFGQG
TKVEI*
SEQ ID NO: 54
EIVLTQSPGTLSLSPGERATLSCRASQSISSSYLAWYQQKPGQAPRLLI
YGASSRATGIPDRFSGSGSGTDFTLTISRLEPEDFAVYYCQQYEAFGQG
TKVEI*
*K deleted in a post translational modification.

In one embodiment the VH sequence is SEQ ID NO: 48 (or a sequence at least 95% identical thereto) and the VL sequence is SEQ ID NO: 52, SEQ ID NO: 53, SEQ ID NO: 54 (or a sequence at least 95% identical to any one of the same).

In one embodiment the VH sequence is SEQ ID NO: 49 (or a sequence at least 95% identical thereto) and the VL sequence is SEQ ID NO: 52, SEQ ID NO: 53, SEQ ID NO: 54 (or a sequence at least 95% identical to any one of the same).

In one embodiment the VH sequence is SEQ ID NO: 50 (or a sequence at least 95% identical thereto) and the VL sequence is SEQ ID NO: 52, SEQ ID NO: 53, SEQ ID NO: 54 (or a sequence at least 95% identical to any one of the same).

In one embodiment the VH sequence is SEQ ID NO: 51 (or a sequence at least 95% identical thereto) and the VL sequence is SEQ ID NO: 52, SEQ ID NO: 53, SEQ ID NO: 54 (or a sequence at least 95% identical to any one of the same).

In one embodiment the VL sequence is SEQ ID NO: 52 (or a sequence at least 95% identical thereto) and the VH sequence is SEQ ID NO: 48, SEQ ID NO: 49, SEQ ID NO: 50 or SEQ ID NO: 51. (or a sequence at least 95% identical to any one of the same).

In one embodiment the VL sequence is SEQ ID NO: 53 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VL sequence is SEQ ID NO: 54 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VH sequence is SEQ ID NO: 51 (or a sequence at least 95% identical thereto) and the VL sequence is SEQ ID NO: 53 ((or a sequence at least 95% identical thereto). Variable region as employed herein refers to the region in an antibody chain comprising the CDRs and a suitable framework.

In one embodiment the heavy chain comprises a sequence independently selected from the group comprising SEQ ID NO: 55, SEQ ID NO: 56, SEQ ID NO: 57, SEQ ID NO: 58, SEQ ID NO: 59 and SEQ ID NO: 60, or a sequence at least 95% identical to any one of the same.

Each SEQ ID NO: 55 to 60 have a post translational modification, which is deletion of K at the C terminal. SEQ ID NO: 55 to 60 are disclosed as SEQ ID NO: 56 to 61 in WO2020/197502, incorporated herein by reference.

In one embodiment the light chain is independently selected from the group comprising SEQ ID NO: 61: SEQ ID NO: 62 and SEQ ID NO: 63 or a sequence at least 95% identical to any one of the same.

SEQ ID NO: 61, 62 and 63 herein are disclosed in WO2020/197502, incorporated herein by reference, as SEQ ID NO: 62, 63 and 55.

In one embodiment the heavy chain is independently selected from SEQ ID NO: 55, 56, 57, 58, 59, and 60 (or a sequence at least 95% identical to any one of the same) and the light chain is independently selected from SEQ ID NO: 61, 62, and 63 (or a sequence at least 95% identical to any one of the same).

In one embodiment the heavy chain is SEQ ID NO: 55 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 56 (or a sequence at least 95% identical thereto) and the light chain is independently selected from SEQ ID NO: 61, 62 and 64 (or a sequence at least 95% identical to any one of the same).

In one embodiment the heavy chain is SEQ ID NO: 57 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 58 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 59 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 60 (or a sequence at least 95% identical thereto) and the light chain is independently selected from SEQ ID NO: 61, 62 and 63 (or a sequence at least 95% identical to any one of the same).

In one embodiment the heavy chain is SEQ ID NO: 58 or 60 (or a sequence at least 95% identical to any one of the same) and a light chain with the sequence shown in SEQ ID NO: 61 (or a sequence at least 95% identical thereto).

In one embodiment the heavy chain is SEQ ID NO: 58 (or a sequence at least 95% identical to any one of the same) and a light chain with the sequence shown in SEQ ID NO: 61 (or a sequence at least 95% identical thereto).

In one embodiment the heavy chain is SEQ ID NO: 60 (or a sequence at least 95% identical to any one of the same) and a light chain with the sequence shown in SEQ ID NO: 61 (or a sequence at least 95% identical thereto).

Derived from as employed herein refers to the fact that the sequence employed or a sequence highly similar to the sequence employed was obtained from the original genetic material, such as the light or heavy chain of an antibody.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

Any discussion of a protein, antibody or amino acid sequence herein will be understood to include any variants of the protein, antibody or amino acid sequence produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” (C-terminal lysine residues of encoded antibodies are often removed during the manufacturing process) and/or have part or all of a signal sequence incompletely processed and, as a consequence, remain at the terminus of the antibody. It is understood that an antibody comprising a particular amino acid sequence or binding fragment thereof may be a heterogeneous mixture of the stated or encoded sequence and/or variants of that stated or encoded sequence or binding fragment thereof.

In one embodiment the present disclosure extends to a sequence explicitly disclosed herein where the C-terminal lysine has been cleaved.

In one embodiment an antibody or binding fragment thereof, employed in a formulation of the present disclosure is humanised.

Humanised (which include CDR-grafted antibodies) as employed herein refers to molecules having one or more complementarity determining regions (CDRs) from a non-human species and a framework region from a human immunoglobulin molecule (see, for example U.S. Pat. No. 5,585,089; WO91/09967). It will be appreciated that it may only be necessary to transfer the specificity determining residues of the CDRs rather than the entire CDR (see for example, Kashmiri et al 2005, Methods, 36, 25-34). Humanised antibodies may optionally further comprise one or more framework residues derived from the non-human species from which the CDRs were derived. For a review, see Vaughan et al, Nature Biotechnology, 16, 535-539, 1998.

When the CDRs or specificity determining residues are grafted, any appropriate acceptor variable region framework sequence may be used having regard to the class/type of the donor antibody from which the CDRs are derived, including mouse, primate and human framework regions. Examples of human frameworks which can be used in the present invention are KOL, NEWM, REI, EU, TUR, TEI, LAY and POM (Kabat et al). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody employed in the present invention, the acceptor heavy and light chains do not necessarily need to be derived from the same antibody and may, if desired, comprise composite chains having framework regions derived from different chains.

The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

In one embodiment the anti-IL13R antibodies of the present disclosure are fully human, in particular one or more of the variable domains are fully human.

Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, U.S. Pat. Nos. 5,545,806, 5,569,825, 5,625,126, 5,633,425, 5,661,016, 5,770,429, EP0438474 and EP0463151.

Thus, the presently disclosed anti-IL13R antibody may comprise one or more constant regions, such as a naturally occurring constant domain or a derivate of a naturally occurring domain. A derivative of a naturally occurring domain as employed herein is intended to refer to where one, two, three, four or five amino acids in a naturally occurring sequence have been replaced or deleted, for example to optimize the properties of the domain such as by eliminating undesirable properties but wherein the characterizing feature(s) of the domain is/are retained.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or indirectly including via a coupling agent to the effector molecule. Techniques for conjugating such effector molecules to antibodies are well known in the art (see, Hellstrom et al, Controlled Drug Delivery, 2nd Ed., Robinson et al, eds., 1987, pp. 623-53; Thorpe et al, 1982, Immunol. Rev., 62:119-58 and Dubowchik et al, 1999, Pharmacology and Therapeutics, 83, 67-123). Particular chemical procedures include, for example, those described in WO93/06231, WO92/22583, WO89/00195, WO89/01476 and WO03/031581. Alternatively, where the effector molecule is a protein or polypeptide the linkage may be achieved using recombinant DNA procedures, for example as described in WO86/01533 and EP0392745.

The term effector molecule as used herein includes, for example, biologically active proteins, for example enzymes, other antibody or antibody fragments, synthetic or naturally occurring polymers, nucleic acids and fragments thereof eg DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U54,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 1251, 1311, 111In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U55,219,996; U55,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

In patients with cancer, such as breast cancer, cancer related lymphedema (BCRL), the formulation of the present disclosure may prevent lymphedema-associated effects, such as fibrosis, hyperkeratosis, the deposition of fibroadipose tissue, fluid accumulation, limb swelling, reduction of skin elasticity, and pain. By reducing the excess volume, said formulation may improve lymphatic and, for example limb functions.

The development of lymphedema after lymphatic injury is associated with tissue inflammation, the infiltration of CD4-positive cells and their differentiation to the type 2 helper T-cell (Th2) phenotype. Th2 cells produce IL-4 and IL-13 that play a key role in the development of lymphedema-associated symptoms as well as other Th2-mediated diseases.

In one embodiment the formulation herein is administered in combination with another therapy.

“In combination” as employed herein is intended to encompass where the anti-IL13R antibody is administered before, concurrently with another therapy or after another therapy, as the same or different formulations. Thus, combination is where the pharmacological effect of a first therapy exists at the same as the existence of a pharmacological effect of second therapy in the body and/or the two therapies are part of treatment plan designed to be employed together.

Therapeutic dose as employed herein refers to the amount of the anti-IL13R antibody, such as ASLAN004 that is suitable for achieving the intended therapeutic effect when employed in a suitable treatment regimen, for example ameliorates symptoms or conditions of a disease, in particular without eliciting dose limiting side effects. Suitable therapeutic doses are generally a balance between therapeutic effect and tolerable toxicity, for example where the side-effect and toxicity are tolerable given the benefit achieved by the therapy.

In one embodiment a formulation according to the present disclosure (including a formulation comprising same) is administered monthly, for example in a treatment cycle or as maintenance therapy.

Unit dose as used herein generally refers to a product comprising the amount of anti-IL13R antibody or binding fragment thereof of the present disclosure that is administered in a single dose including any overage.

A unit dose of the presently claimed anti-IL13R antibody or antigen binding fragment thereof may refer to the marketed form of the product, such as a formulation of the anti-IL13R antibody or binding fragment thereof, wherein the product is apportioned into the amount of anti-IL13R antibody that is required for a single dose. Thus, the manufacturer is able to determine and control the exact amount of anti-13R antibody or binding fragment thereof to be included in each unit dose.

The product may be in various forms, familiar to the skilled addressee, such as vials, ampoules, infusion bags or a device (including an auto-injection device).

The exact amount as employed herein refers to the amount to be administer as a dose to the patient and any overage.

In one embodiment, the unit dose or unit doses are for use according to a method of the present disclosure.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference. Any embodiments specifically and explicitly recited herein may form the basis of a disclaimer either alone or in combination with one or more further embodiments.

The background section of this specification contains relevant technical information and may be used as basis for amendment.

Subject headings herein are employed to divide the document into sections and are not intended to be used to construe the meaning of the disclosure provided herein.

The present specification claims priority from SG10202102087T (filed 1 Mar. 2021); SG 10202110690S (filed 27 Sep. 2021) both incorporated herein by reference. These applications may be used as basis for corrections to the present specification, especially in respect of sequences disclosed therein.

The present invention is further described by way of illustration only in the following examples.

### BRIEF DESCRIPTION OF FIGURES

FIG. 1A Table showing demographics of full analysis set

FIG. 1B Table showing baseline disease characteristics of full analysis set

FIG. 1C Table showing baseline disease characteristics of Evaluable for Efficacy set (EES) FIG. 2A Table showing % change from baseline in EASI score at Day 57 for EES.

FIG. 2B Graph showing % change from baseline in EASI score at Day 57 for EES (ASLAN004 200 mg, 400 mg and 600 mg)

FIG. 2C Graph showing % change from baseline in EASI score at Day 57 for EES (ASLAN004 low dose and high doses)

FIG. 3A Table showing % change from baseline in EASI score at Day 29 for EES

FIG. 3B Graph showing % change from baseline in EASI score at Day 29 for EES (ASLAN004 200 mg, 400 mg and 600 mg)

FIG. 4A Graph showing % change in EASI score over time for EES (ASLAN004 200 mg, 400 mg and 600 mg)

FIG. 4B Graph showing % change in EASI score over time for EES (ASLAN004 low and high dose)

FIG. 5A Graph showing % change in baseline in EASI score over time for EES (Placebo)

FIG. 5B Graph showing % change in baseline in EASI score over time for EES (ASLAN004 200 mg)

FIG. 5C Graph showing % change in baseline in EASI score over time for EES (ASLAN004 400 mg)

FIG. 5D Graph showing % change in baseline in EASI score over time for EES (ASLAN004 600 mg)

FIG. 6A Table showing Day 57 sensitivity analysis in the modified intention to treat (mITT)

FIG. 6B Graph showing Day 57 sensitivity analysis in the mITT (ASLAN004 200 mg, 400 mg and 600 mg)

FIG. 6C Graph showing Day 57 sensitivity analysis in the mITT (ASLAN004 low and high dose)

FIG. 7A Summary table showing EASI 50, EASI 75, EASI 90 at Day 57 for EES FIG. 7B Graph showing EASI 50 at Day 57 for EES (ASLAN004 200 mg, 400 mg and 600 mg)

FIG. 7C Graph showing EASI 50 at Day 57 for EES (ASLAN004 low and high dose)

FIG. 7D Graph showing EASI 75 at Day 57 for EES (ASLAN004 200 mg, 400 mg and 600 mg)

FIG. 7E Graph showing EASI 75 at Day 57 for EES (ASLAN004 low and high dose)

FIG. 7F Graph showing EASI 90 at Day 57 for EES (ASLAN004 200 mg, 400 mg and 600 mg)

FIG. 7G Graph showing EASI 90 at Day 57 for EES (ASLAN004 low and high dose)

FIG. 8A Graph showing proportion of patients achieving EASI 50 FIG. 8B Graph showing proportion of patients achieving EASI 75

FIG. 8C Graph showing proportion of patients achieving EASI 90

FIG. 9 Summary table showing proportion of patients achieving EAS 150, 75, 90 for EES and mITT

FIG. 10A Summary table showing proportion of patients with IGA score of 0 or 1 at Day 57 for ESS

FIG. 10B Graph showing proportion of patients with IGA score of 0 or 1 at Day 57 for ESS

FIG. 10C Graph showing proportion of patients with IGA score of 0 or 1 over time for ESS

FIG. 11 Table showing baseline TARC and IgE levels of patients

FIG. 12A Graph showing average % change from baseline TARC (ASLAN004 200 mg and 400 mg)

FIG. 12B Graph showing average % change from baseline TARC (ASLAN004 400 mg and placebo)

FIG. 12C Graph showing % change from baseline TARC for individual patients (ASLAN004 400 mg)

FIG. 13A Graph showing IgE % change from baseline for ASLAN004 200 mg and 400 mg

FIG. 13B Graph showing average IgE % change from baseline for ASLAN004 200 mg and 400 mg

FIG. 13C Graph showing IgE % change from baseline for individual patients for placebo

FIG. 13D Graph showing IgE % change from baseline for individual patients for ASLAN004 200 mg

FIG. 13E Graph showing IgE % change from baseline for individual patients for ASLAN004 400 mg

FIG. 14 Graph showing comparison between ASLAN004 exposure, EASI, TARC and IgE over time for patients receiving ASLAN004 400 mg

FIG. 15 Table showing comparison between ASLAN004 and Duplilumab

FIG. 16A Table showing patient demographics and baseline characteristics for mITT (modified intention to treat) group

FIG. 16B Table showing patient demographics and baseline characteristics for Excluded site group

FIG. 17A Graph showing TARC/CCL17 median % change from baseline (% CFBL) at week 8 for mITT group

FIG. 17B Graph showing TARC/CCL17 median % CFBL at week 8 for Excluded site group

FIG. 17C Graph showing TARC/CCL17 median % CFBL over time for mITT group

FIG. 18A Graph showing total IgE median % change from baseline at week 8 for mITT group. Day 15 was first post-baseline time point assessed for IgE.

FIG. 18B Graph showing total IgE median % change from baseline over time for mITT group. Day 15 was first post-baseline time point assessed for IgE.

FIG. 19A Graph showing lactate dehydrogenase (LDH) median % change from baseline at week 8 for mITT group.

FIG. 19B Graph showing lactate dehydrogenase (LDH) median % change from baseline at over time for mITT group.

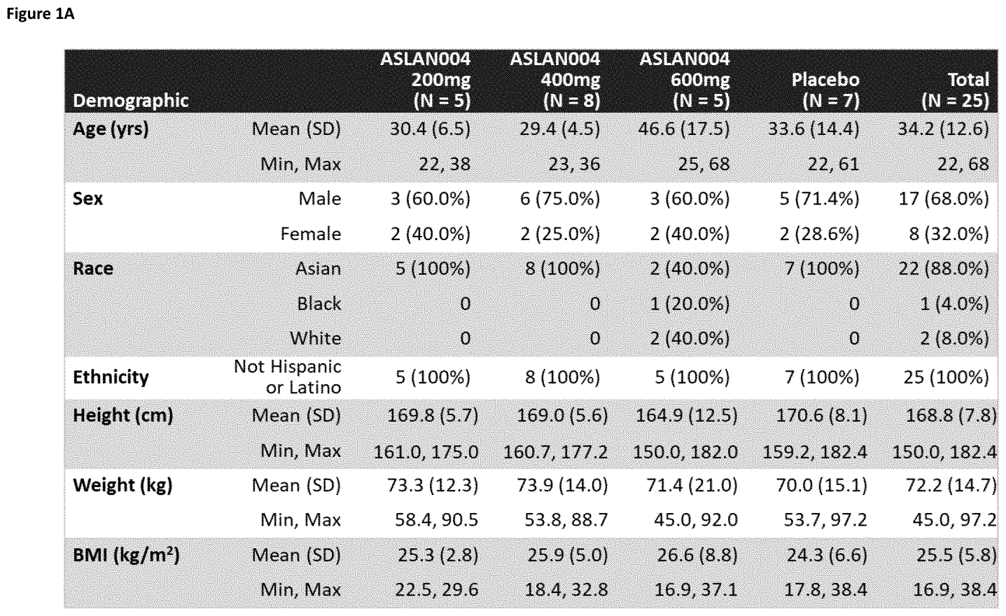

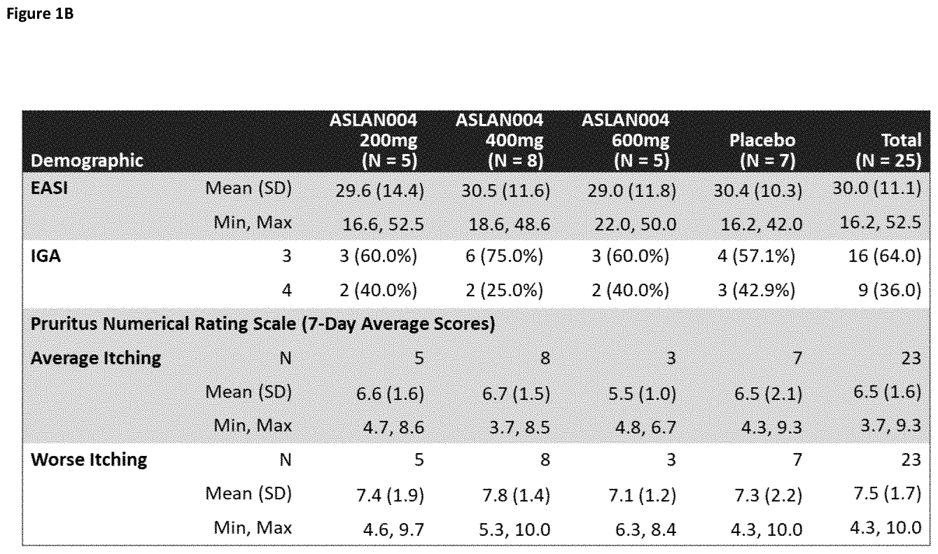

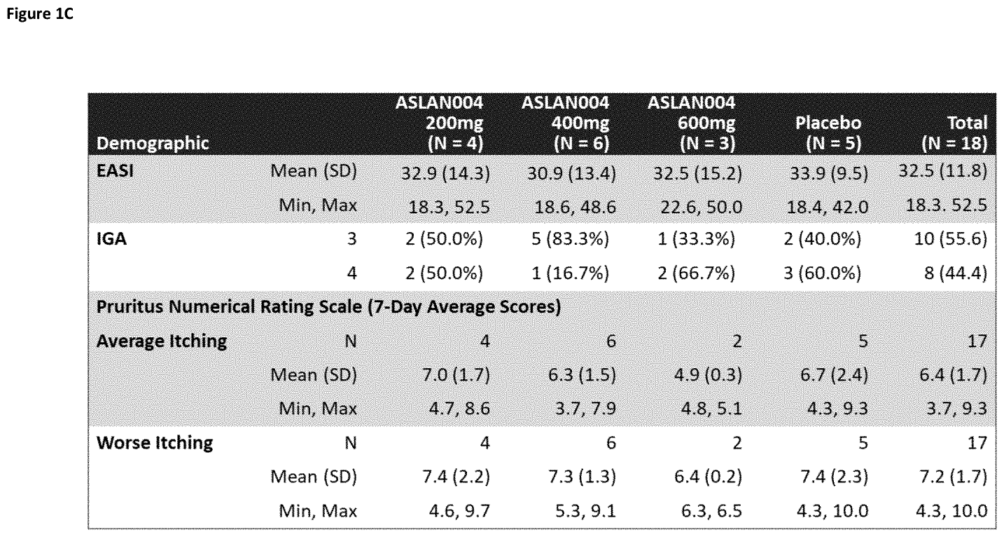

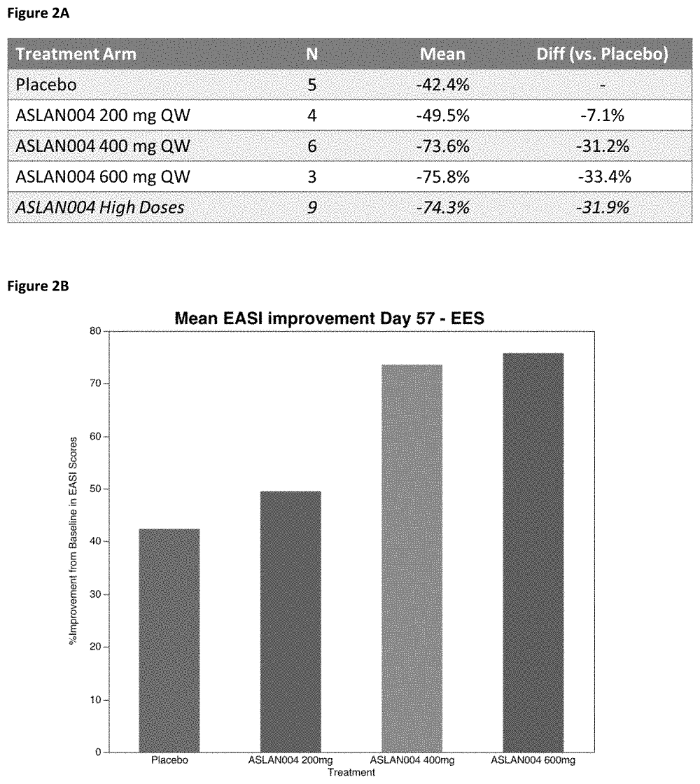

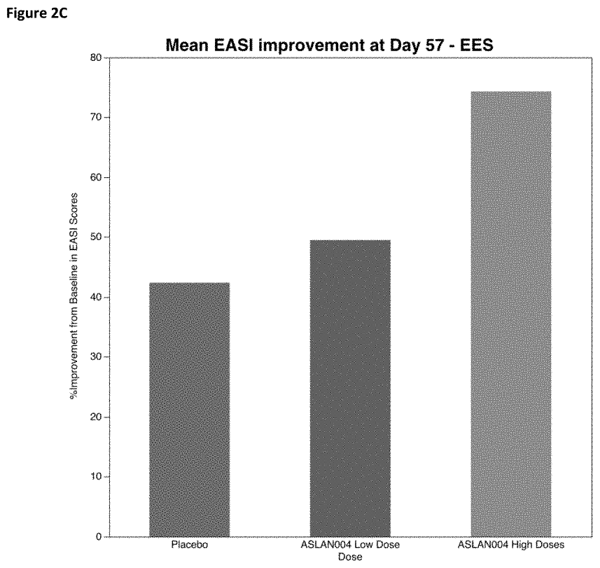

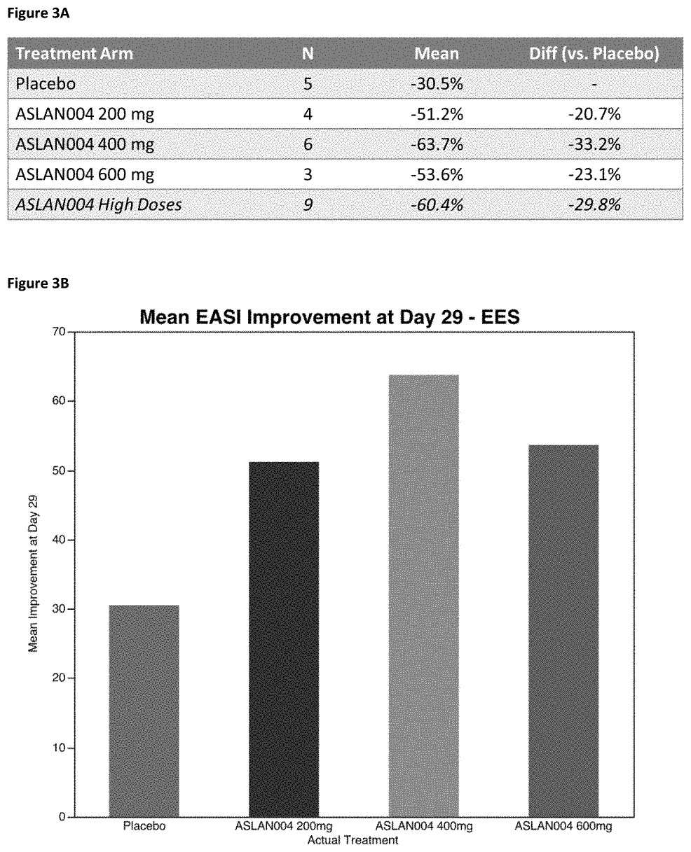

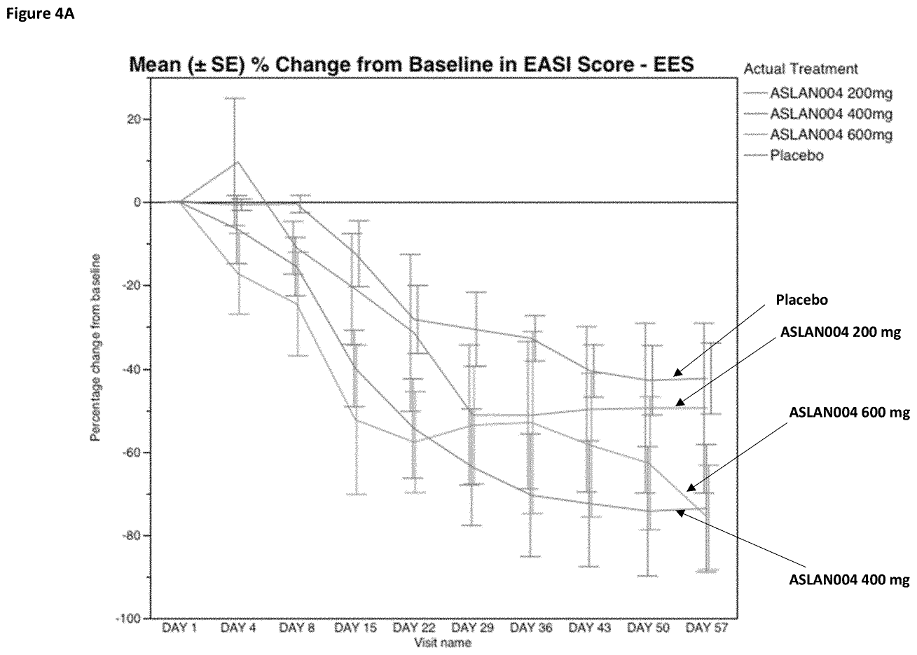

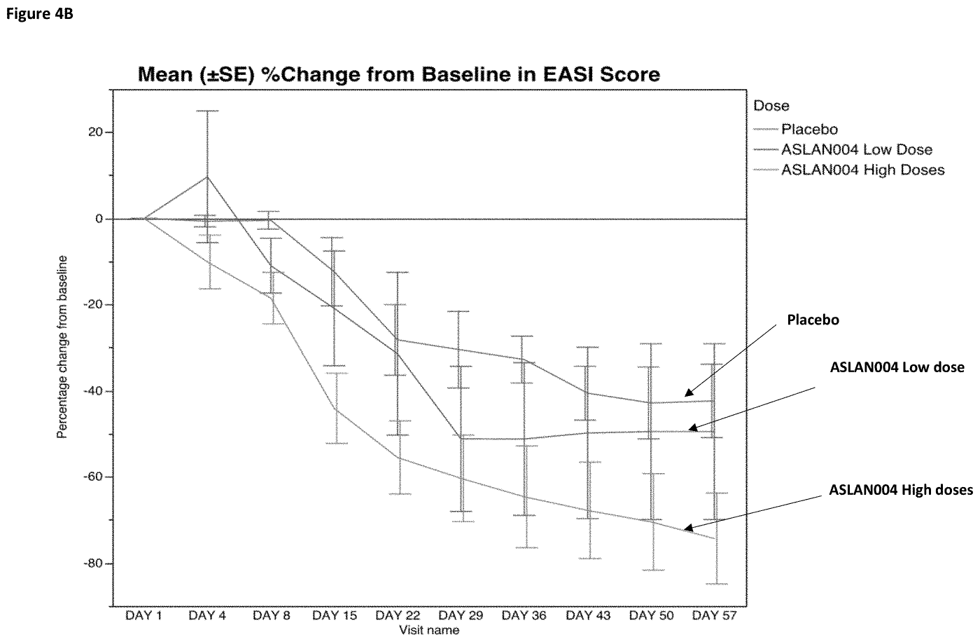

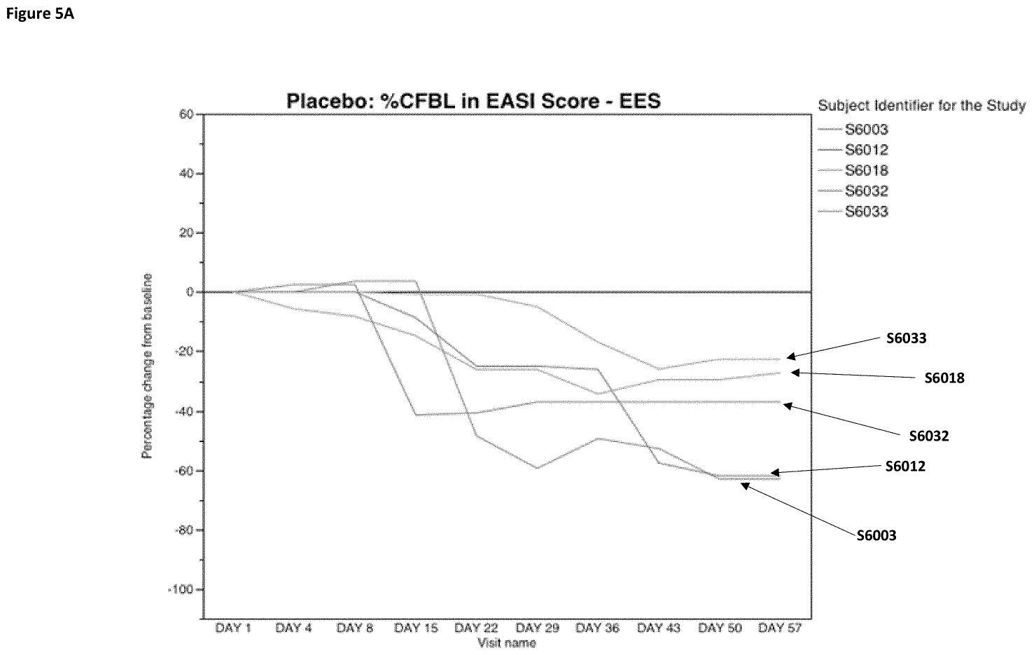

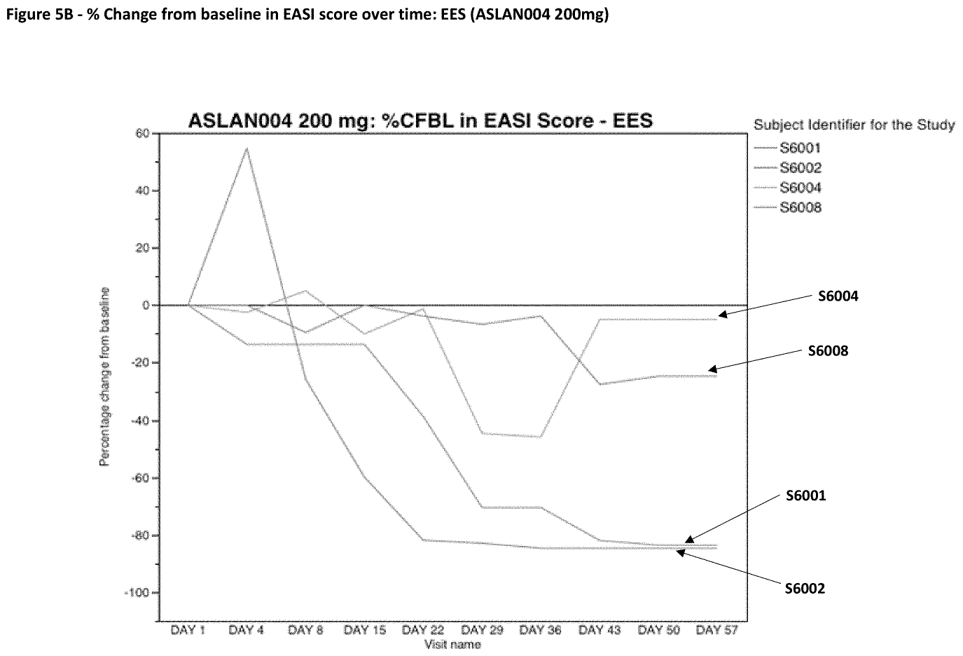

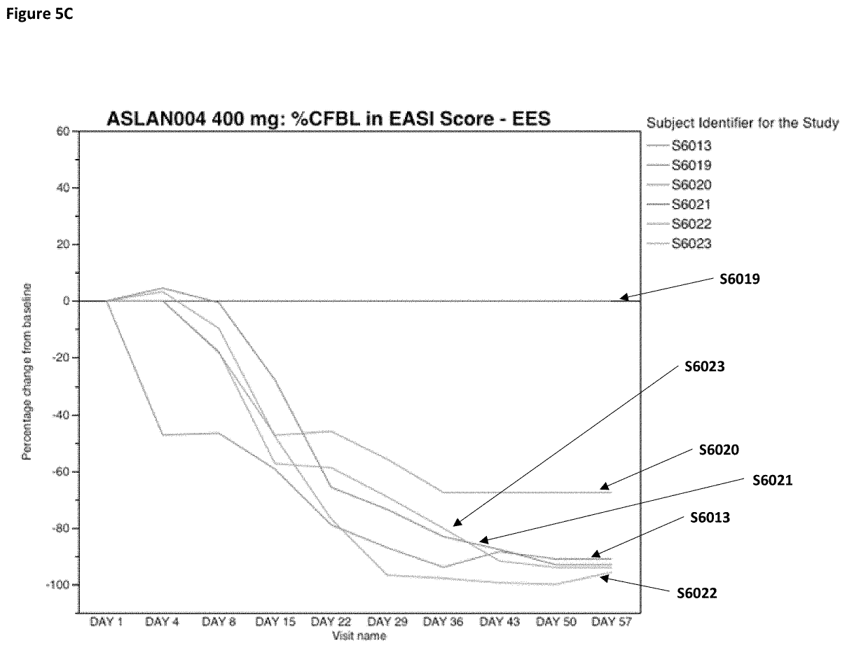

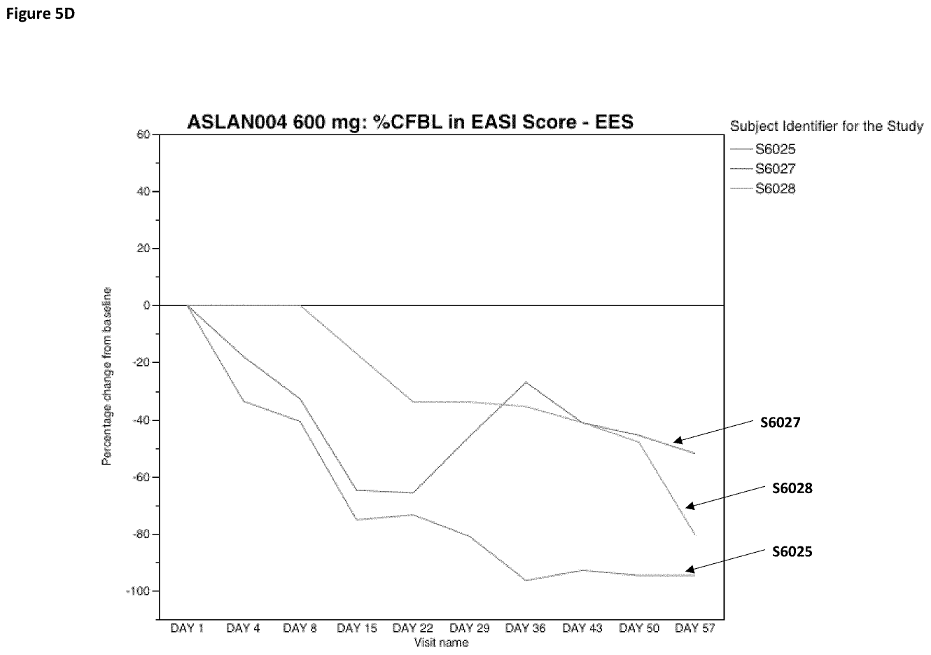

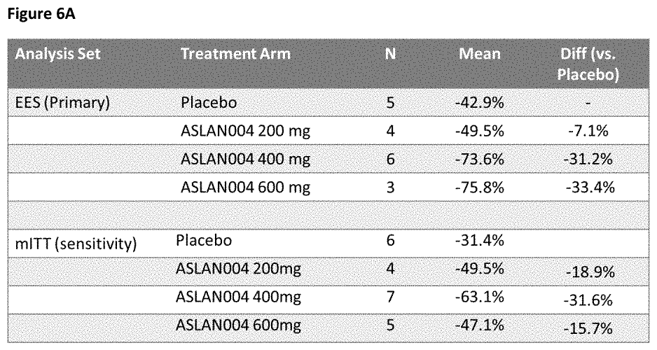

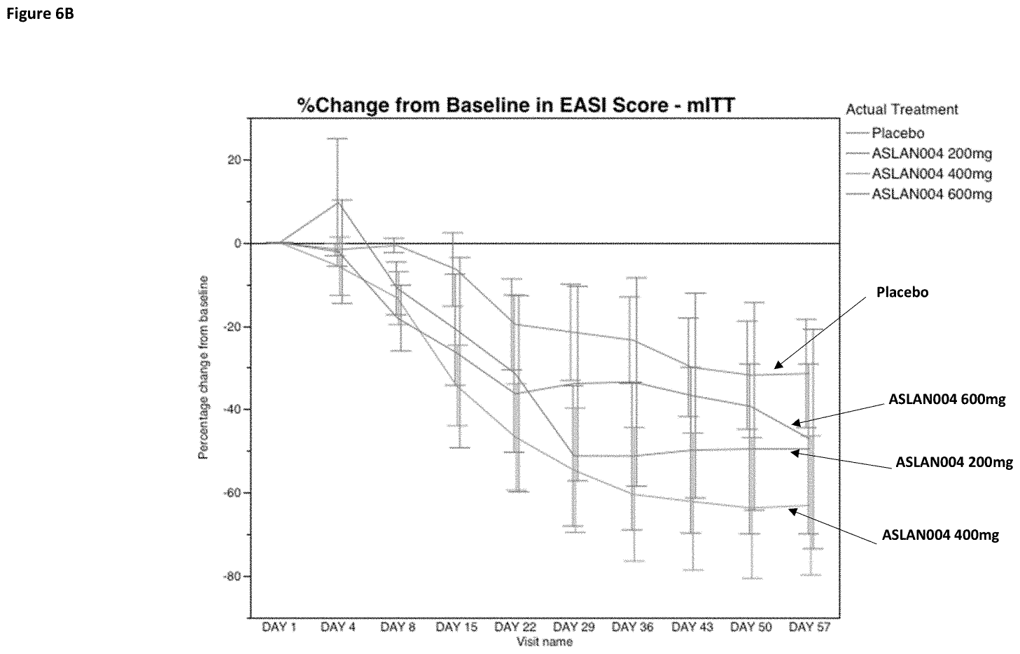

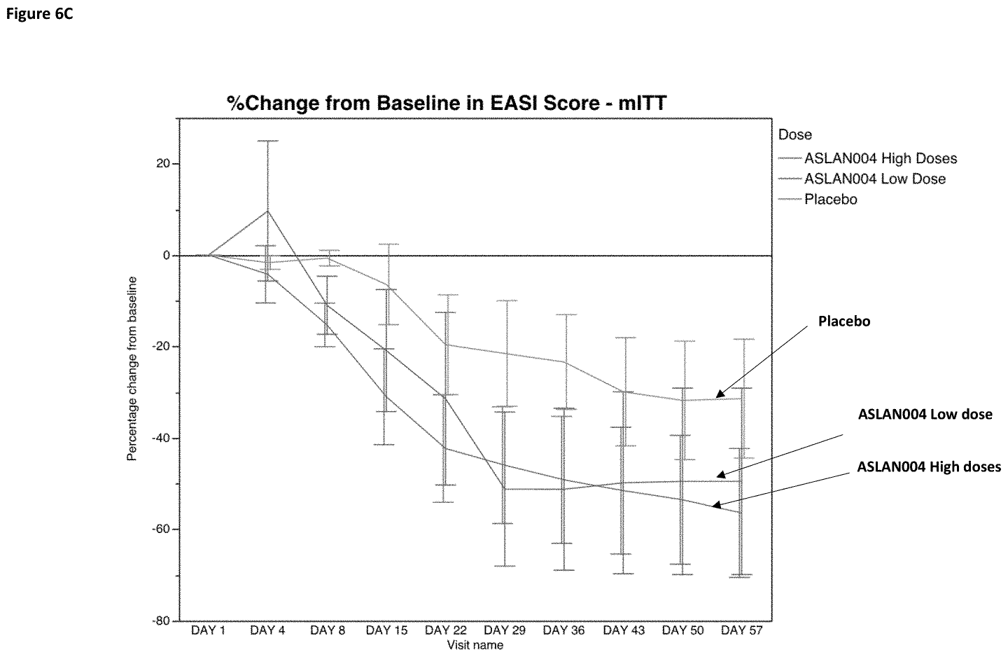

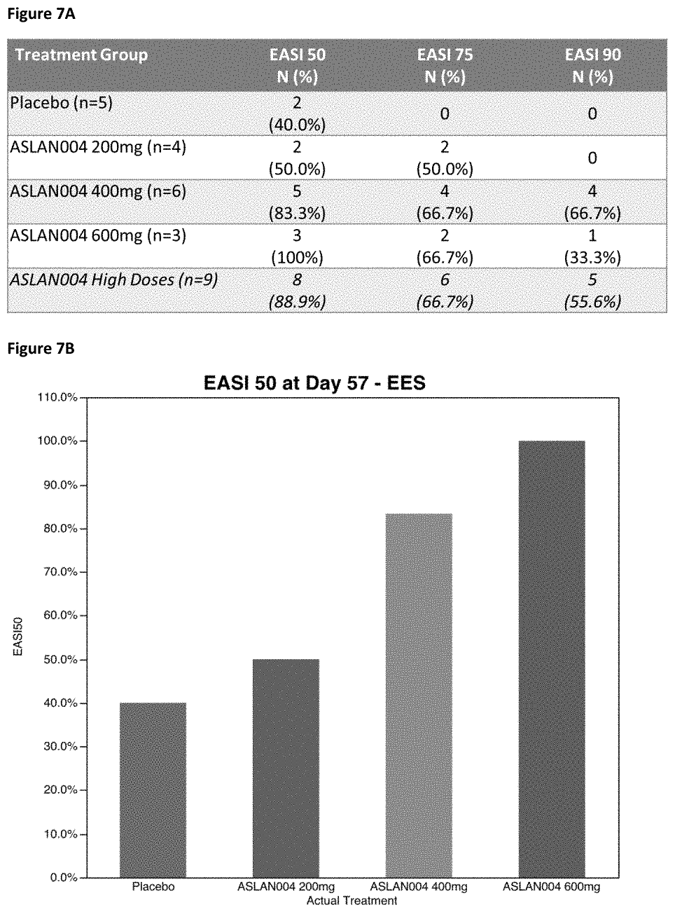

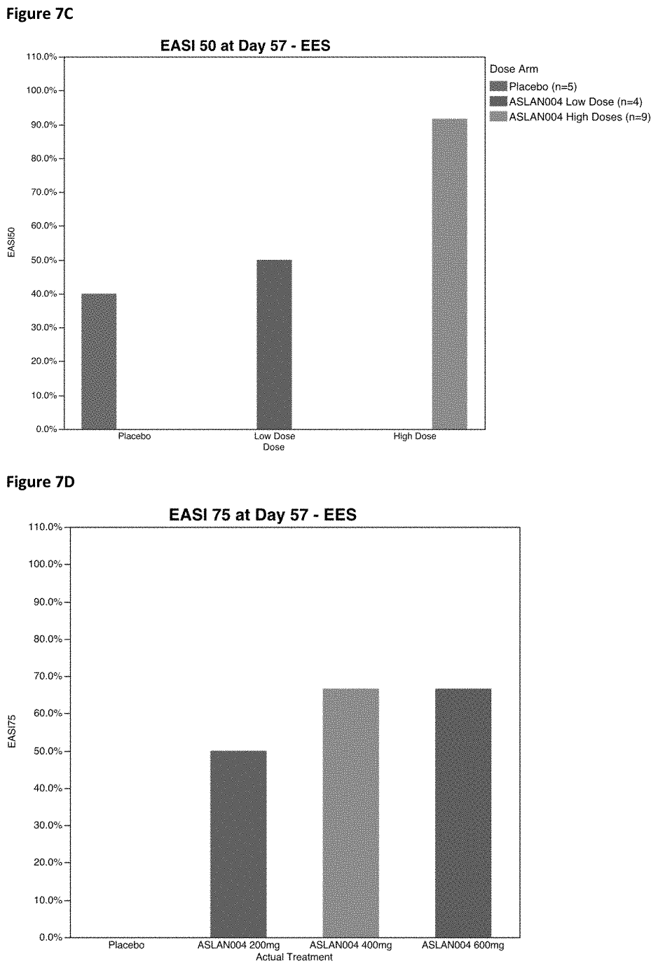

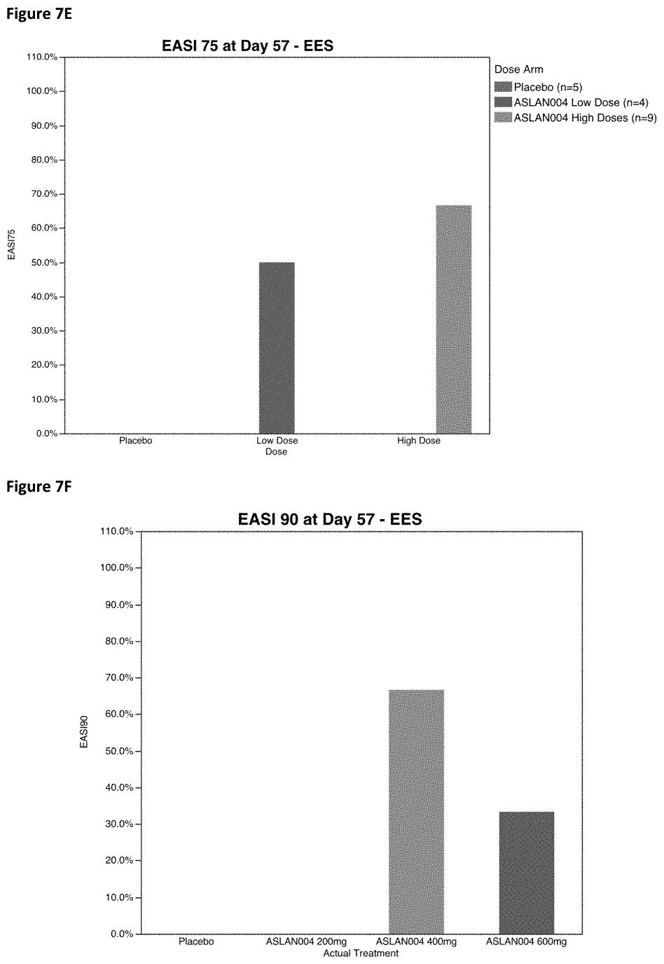

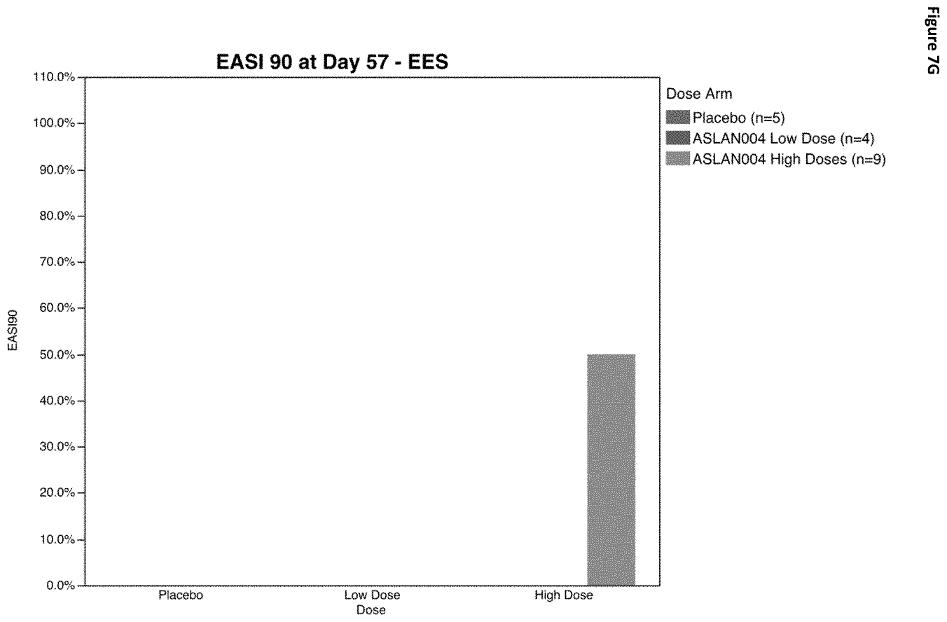

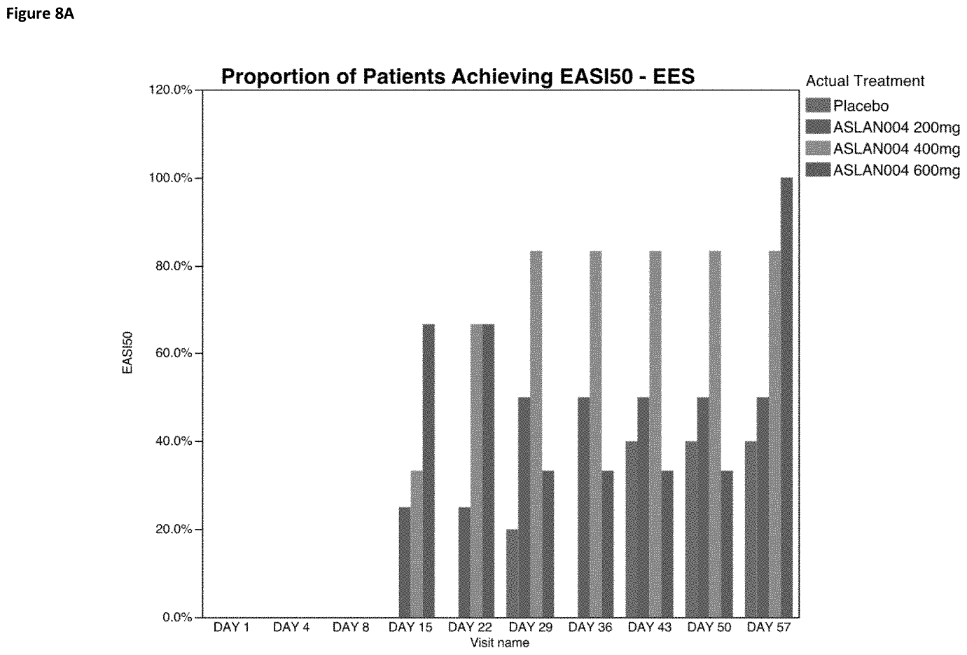

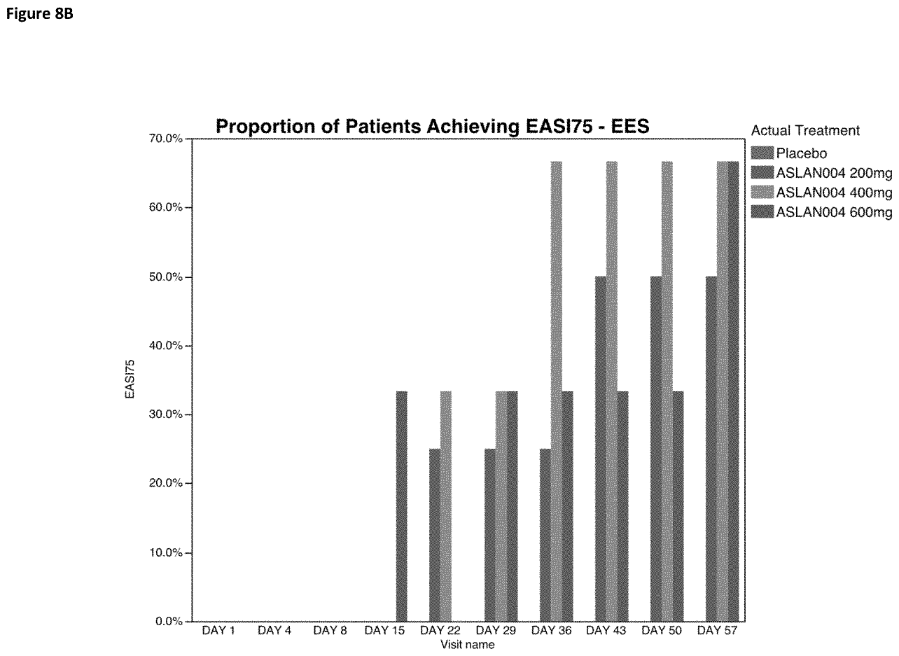

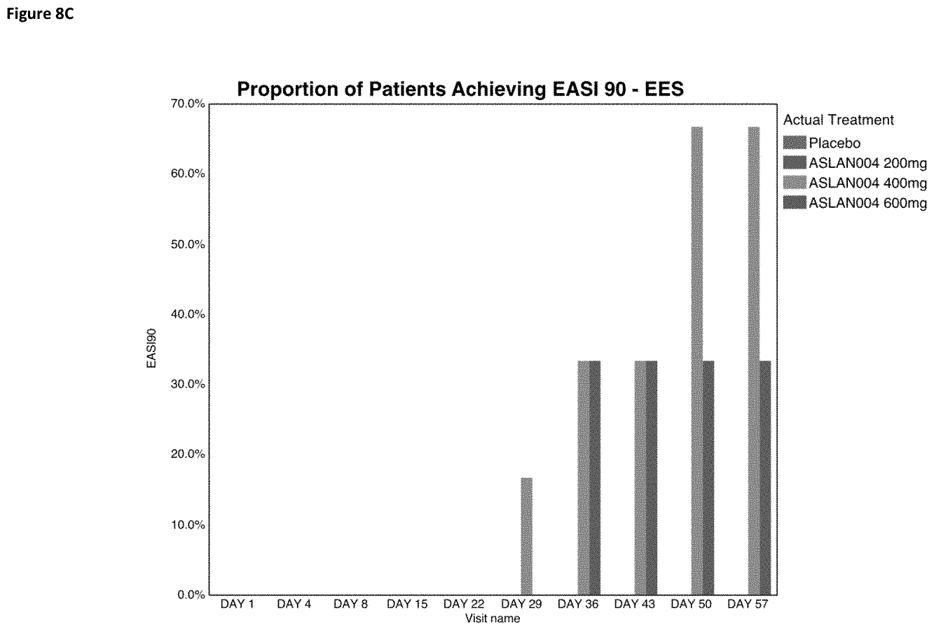

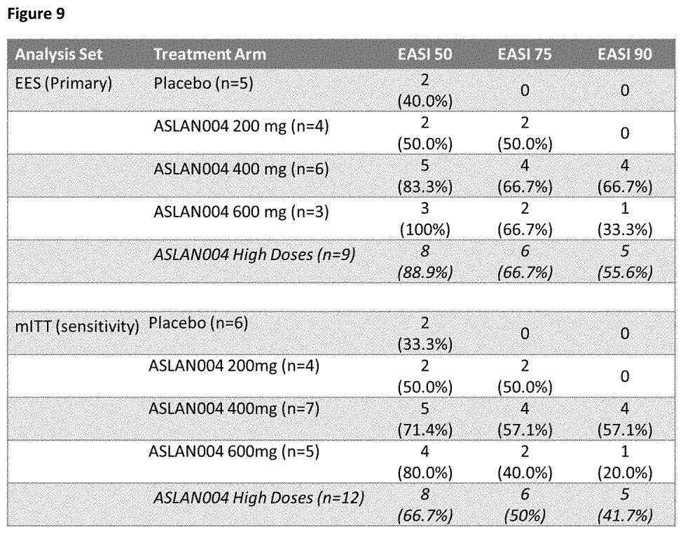

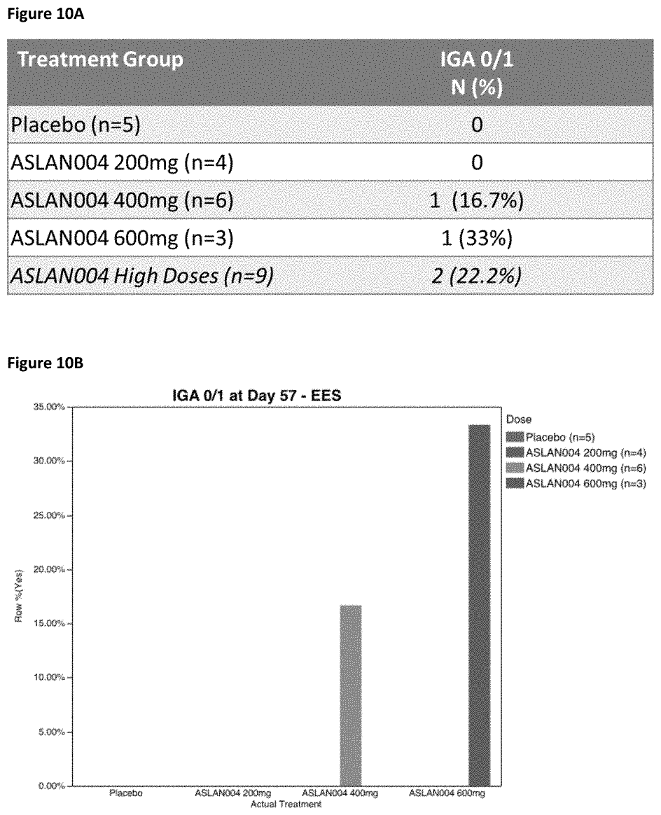

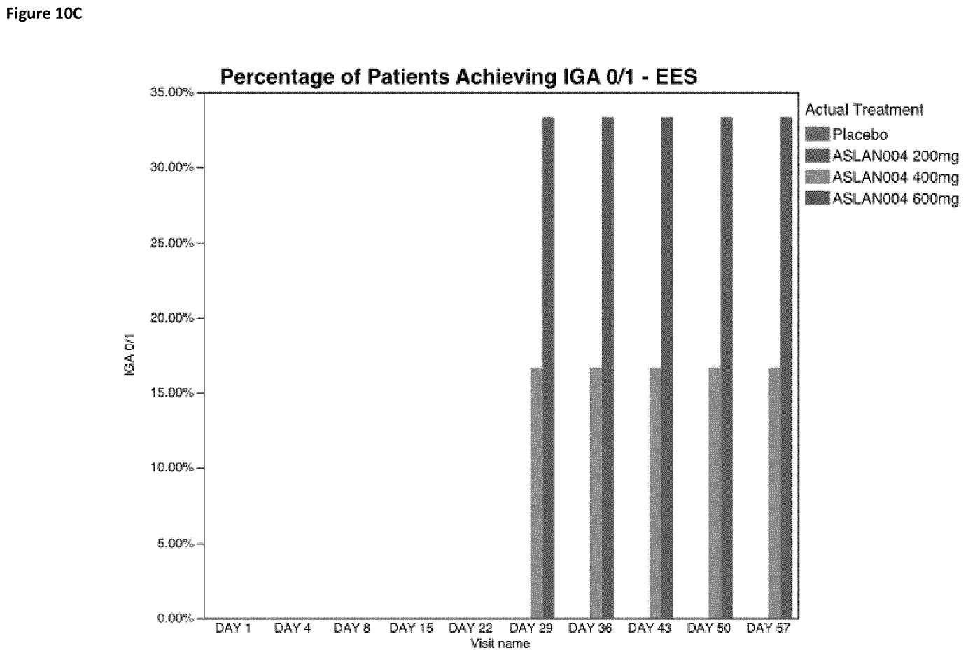

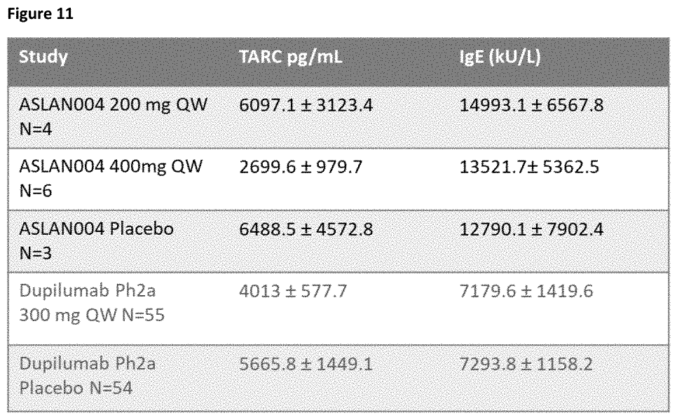

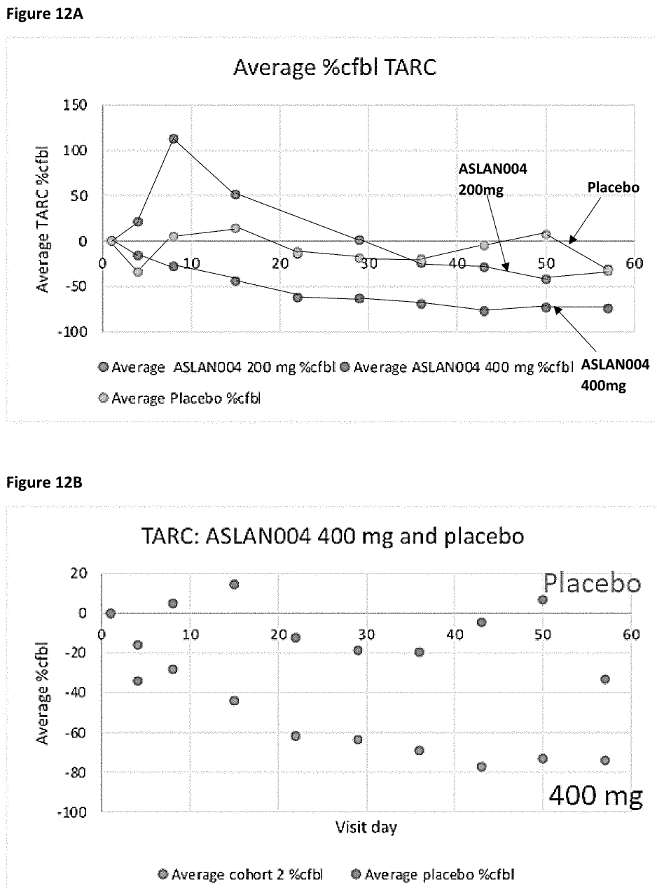

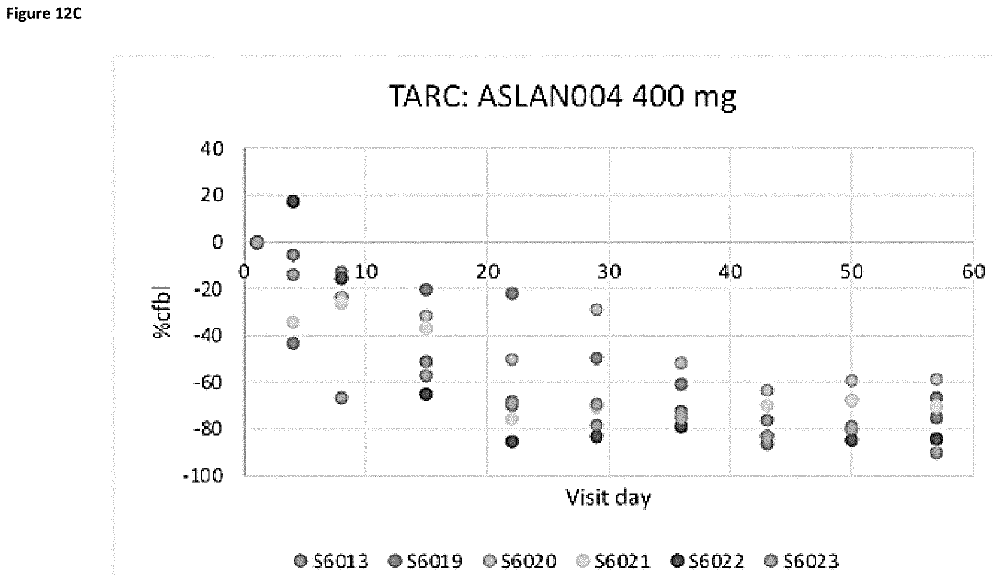

### EXAMPLES

### Example 1 Study Protocol (Initial MAD Escalation)

Patients enrolled in ascending dose cohorts of ASLAN004 (SEQ ID NO: 51, 53 and 59 herein): 200 mg, 400 mg 600 mg. Initially the doses were given QW. Within each cohort, patients were randomized in a 3:1 ratio of ASLAN004: Placebo

TABLE 1
Baseline TARC and IgE

| Study | TARC pg/mL | IgE (kU/L) |
|---|---|---|
| ASLAN004 200 mg | 6097.1 ± 3123.4 | 14993.1 ± 6567.8 |
| QW N = 4 | | |
| ASLAN004 200 mg | 2699.6 ± 979.7 | 13521.7 ± 5362.5 |
| QW N = 6 | | |
| ASLAN004 Placebo | 6488.5 ± 4572.8 | 12790.1 ± 7902.4 |
| N = 3 | | |
| Dupilumab Ph2a | 4013 ± 577.7 | 7179.6 ± 1419.6 |
| 300 mg QW N = 55 | | |
| Dupilumab Ph2a | 5665.8 ± 1449.1 | 7293.8 ± 1158.2 |
| Placebo N = 54 | | |

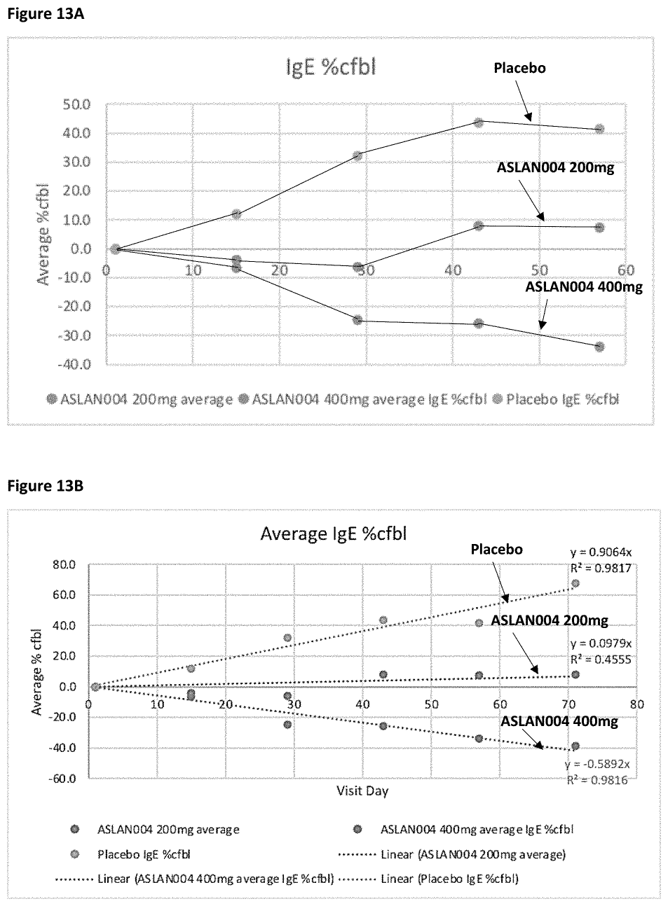

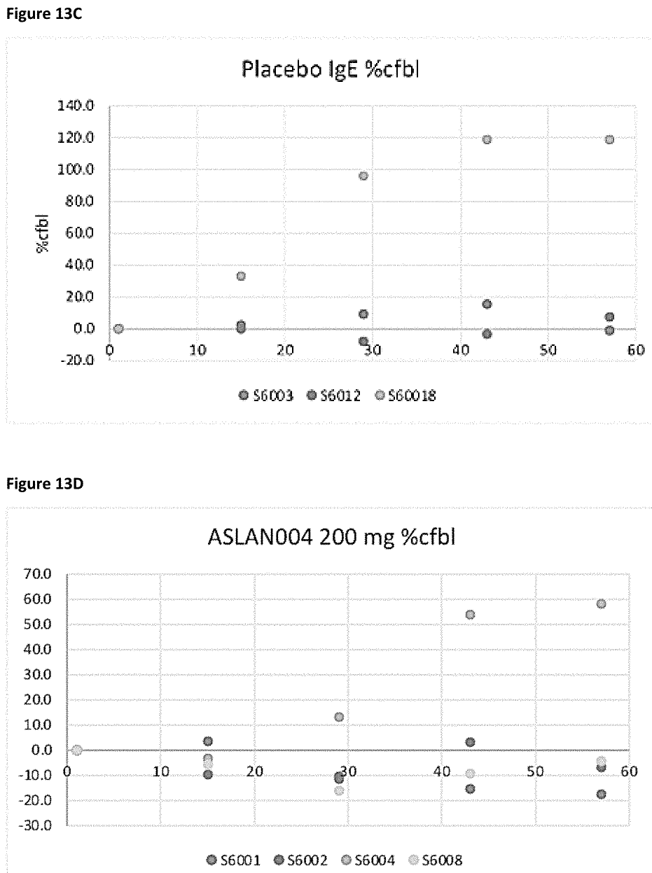

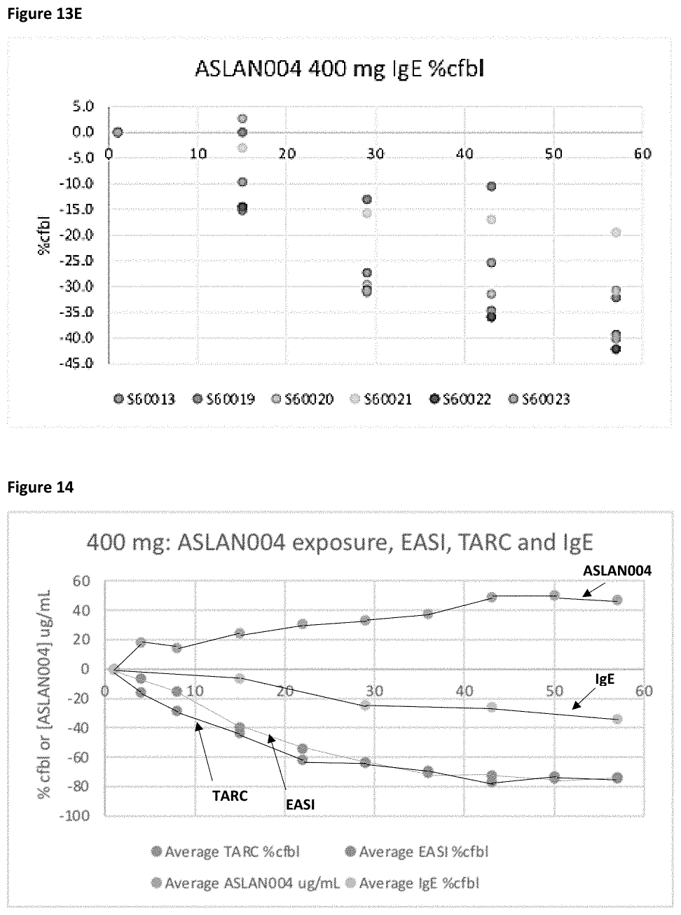

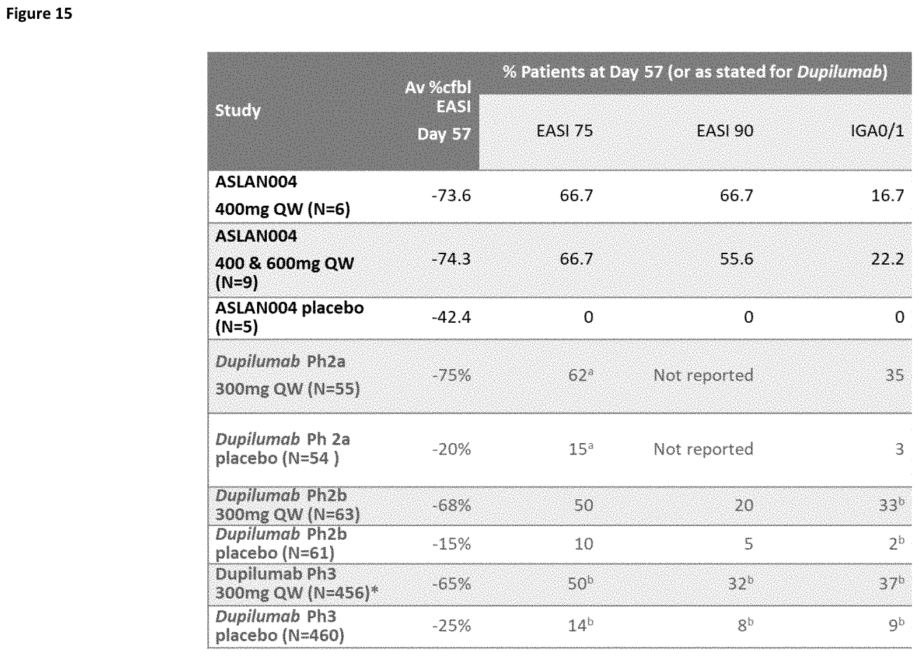

FIG. 13A shows the % change from baseline at day 15, 29, 43 and 57 or all patients receiving treatment. FIG. 13B shows the average % change from baseline over the same period.

FIG. 13C to E shows the data for the individual patients.

TABLE 2
Shows a reduction and −negative number

| | Placebo | Drug |
|---|---|---|
| Study | % cfbl | % cfbl |
| ASLAN004 | 41.6 | −34 |
| 400 mg QW | | |
| Dupilumab | 15 | −35 |
| 300 mg QW Ph2a | | |
| Dupilumab | 10 | −15 |
| 300 mg QW Ph2b/Ph3 | 15 | −30 |

It can be seen that in highly allergic population of patients (IgE 41.6% cfbl) ASLAN004 reduced the IgE levels by −34%. In real terms this is a more significant reduction than the −15 to −30% in a population with lower allergic response in dupilumab.

### Example 2

In the 32 patients that completed at least 29 days of dosing across all sites, defined in the protocol as the efficacy evaluable data set, the average reduction from baseline in EASI at 8 weeks was 73% (n=19) compared to 44% (n=13) for patients on placebo (p=0.007¹).

The proportion of patients with adverse events and treatment-related adverse events were similar across treatment and placebo arms. There were no incidences of conjunctivitis in the expansion cohort

| | RITT (n = 29) | | | ITT (n = 38) | | |
|---|---|---|---|---|---|---|
| | 600 mg | Placebo | p- | 600 mg | Placebo | p- |
| Endpoint | (n = 16) | (n = 13) | value¹ | (n = 22) | (n = 16) | value¹ |
| Mean % change from | −64.9 | −27.2 | 0.021 | −61.3 | −31.9 | 0.023 |
| baseline in EASI | | | | | | |
| EASI-50 (%) | 81.3 | 30.8 | 0.008 | 77.3 | 37.5 | 0.016 |
| EASI-75 (%) | 68.8 | 15.4 | 0.005 | 50.0 | 12.5 | 0.018 |
| EASI-90 (%) | 37.5 | 15.4 | 0.183 | 27.3 | 12.5 | 0.245 |
| IGA 0/1 (%) | 43.8 | 15.4 | 0.107 | 31.8 | 18.8 | 0.301 |
| Mean % change from | −38.6 | −15.3 | 0.051 | −37.1 | −15.7 | 0.032 |
| baseline in peak | | | | | | |
| pruritis Numerical | | | | | | |
| Rating Scale | | | | | | |
| Mean change from | −9.8 | −2.5 | 0.007 | −9.0 | −3.5 | 0.014 |
| baseline in POEM | | | | | | |

¹One-sided p-value

ASLAN004 achieved a statistically significant improvement (p<0.025) versus placebo in the primary efficacy endpoint of percent change from baseline in the Eczema Area Severity Index (EASI), and also showed significant improvements (p<0.05) in other key efficacy endpoints: EASI-50, EASI-75, peak pruritis and the Patient-Oriented Eczema Measure (POEM).

Following discussions with the Data Monitoring Committee prior to unblinding, a Revised ITT population (RITT, n=29) was defined to exclude one study site at which all patients enrolled in the study appeared atypical of moderate-to-severe AD patients based on biomarkers, such as TARC, and patient medical history. In the RITT population, which is more comparable to other published studies in moderate-to-severe AD, ASLAN004 also achieved a statistically significant improvement (p<0.025) versus placebo in percent change from baseline in EASI and showed a greater improvement over placebo in the key efficacy endpoints versus the ITT population.

### Example 3

A range of asthma patients, for example moderate to severe allergic asthma patients, in particular poorly controlled asthma patients can be treated as per Example 1.

### Example 4

A range of eosinophilic esophagitis patients, for example moderate to severe eosinophilic esophagitis patients, in particular poorly controlled asthma patients can be treated as per Example 1.

### Example 5

Elevated serum levels of specific biomarkers are associated with increased disease severity and exacerbations of AD. These biomarkers include thymus and activation regulated chemokine (TARC/CCL17), total immunoglobulin E (IgE), lactate dehydrogenase (LDH).

Reference range levels for these biomarkers in individuals without AD have been reported in the range of:
TARC/CCL17: 200 pg/mL
Total IgE: 150 to 1,000 UI/mL (usually accepted upper limit is between 150 and 300 UI/mL)
LDH: 105 to 333 U/L

TARC/CCL17 is a chemokine involved in developing acute and chronic lesions in AD and serves as a biomarker for disease severity. IgE binds several immune cells and plays a role in the release of inflammatory mediators and antigen presentation in atopic dermatitis. LDH is an enzyme found in most cells and is known to be a marker of inflammation, but it also has been shown to correlate with levels of TARC/CCL17 and total IgE in patients with AD.

The objective of this study was to investigate the pharmacodynamic (PD) effect of weekly eblasakimab administration on biomarkers of allergic inflammation (TARC/CCL17, total IgE and LDH) in patients with moderate-to-severe AD.

### Methods

A placebo-controlled, double-blind, multiple ascending dose phase 1b study was conducted in which patients with moderate-to-severe AD received either eblasakimab (200, 400, 600 mg) or placebo administered subcutaneously once weekly for 8 weeks, with a 12-week safety follow-up period.

Key inclusion criteria were:
Chronic AD present for years before screening visit
Eczema Area and Severity Index score at screening and baseline
Investigator Global Assessment score (scale of 0 to 4) at screening and baseline
≥10% body surface area of AD involvement at screening and baseline

Blood samples were taken at pre-specified time points to assess levels of TARC/CCL17, total IgE, and LDH:
TARC/CCL17 levels were measured in duplicate using an enzyme-linked immunosorbent assay (ELISA) assay.
Total IgE concentrations were determined in duplicate using the validated ImmunoCap
Total IgE assay (Thermo Fisher Scientific).
LDH was determined as part of routine laboratory safety monitoring.
Samples were analyzed and reported by a local testing laboratory following their protocols and procedures.

Analyses were performed on 3 populations:
Modified intent-to-treat population (mITT)—the intent-to-treat population excluding 9 patients at one clinical site that were markedly different from the mITT set at baseline (see next bullet point) and were excluded in a pre-specified sensitivity analysis that was defined prior to unblinding.
Excluded site—Further data are presented from a prespecified subgroup of patients from an excluded site with atypical AD. This excluded site consisted of the 9 patients noted above at one clinical site that were markedly different from the mITT set at baseline due to characteristics including older age, lower scores on AD measures, lack of atopic disease history, and higher rates of diabetes and hypertension.
Safety population—all randomized patients who received at least 1 dose of study drug.

Mean percent changes from baseline in levels of each biomarker are reported:
Inferential statistical analysis was performed for TARC/CCL17 and IgE for 600 mg vs placebo groups at week 8 only using an ANCOVA model fitting the Week 8% CFBL as the response variable, and treatment (eblasakimab or placebo) and the baseline biomarker score as covariates, with a prespecified 1-sided 5% significance level.
Results for LDH as well as for the 200 and 400 mg groups for TARC and IgE were descriptively described due to small sample size.

### Results

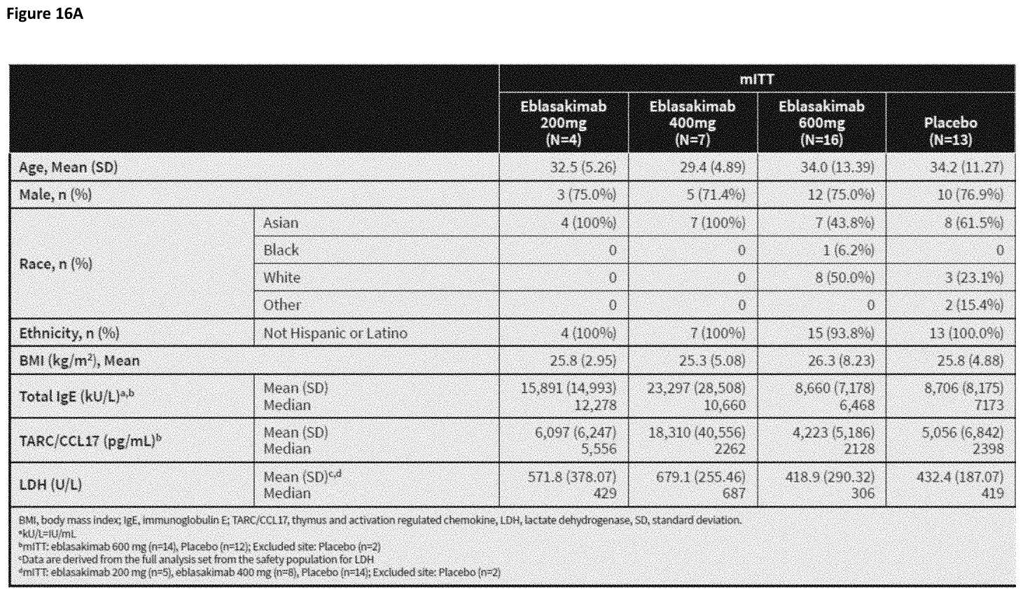

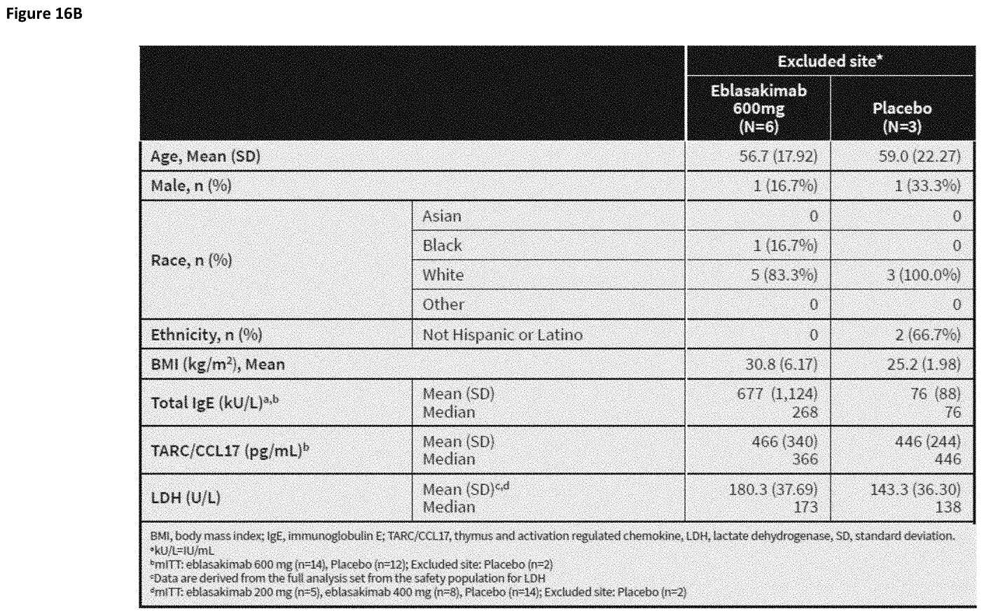

A total of 40 patients were included in the mITT population analysis and received either eblasakimab at 200 mg (N=4), 400 mg (N=7), 600 mg (N=16), or placebo (N=13) (FIG. 16A).

Patient demographics and baseline characteristics for the mITT population were generally similar across dose cohorts, with a slightly younger population in the 400 mg group, a higher proportion of Asian patients in the 200 mg and 400 mg groups, and some differences in the level of elevation of biomarkers, particularly for TARC/CCL17 and total IgE (FIG. 16A).

The Excluded site set was markedly different from the mITT set at baseline with substantially lower serum TARC/CCL17 (FIG. 16B), serum IgE (FIG. 16B), and EASI scores (data not shown) showing lower extent and severity of disease. Other notable differences included older age and lower IGA and BSA. Participants at this site had no atopic disease history but reported other comorbidities including diabetes and hypertension.

Baseline biomarker levels from the Excluded site were, on average, within the reference range for individuals without AD, except for TARC/CCL17 levels, which were slightly elevated (FIG. 16A).

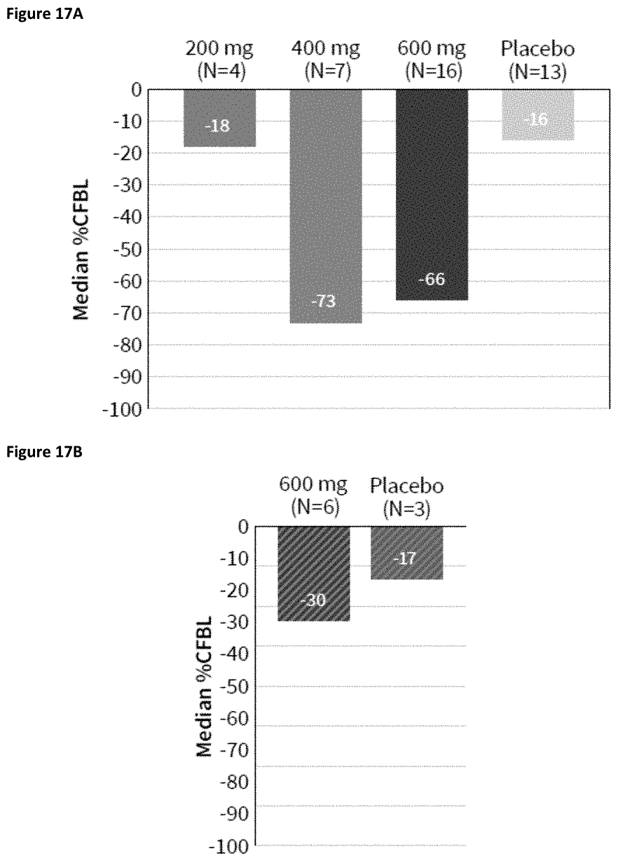

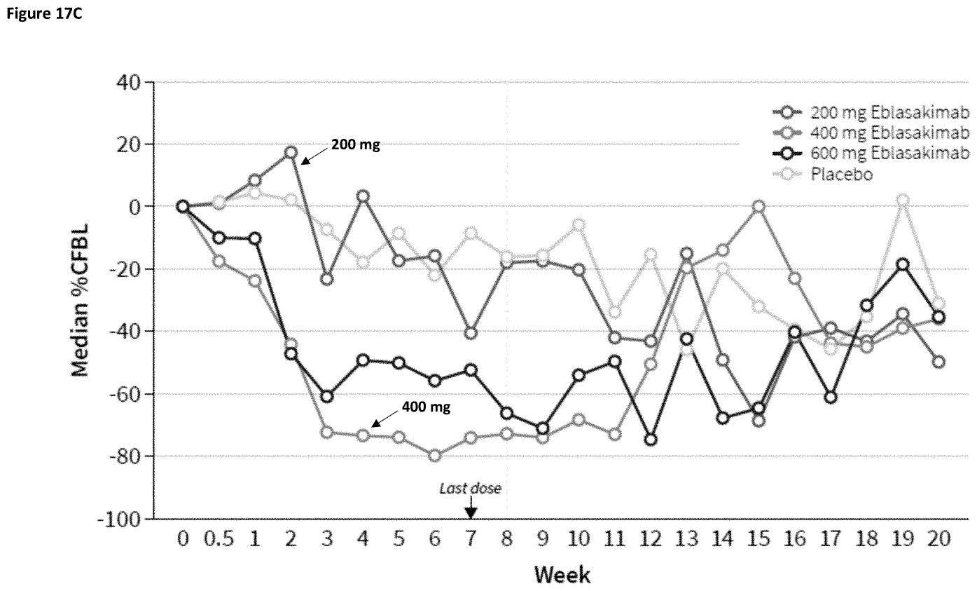

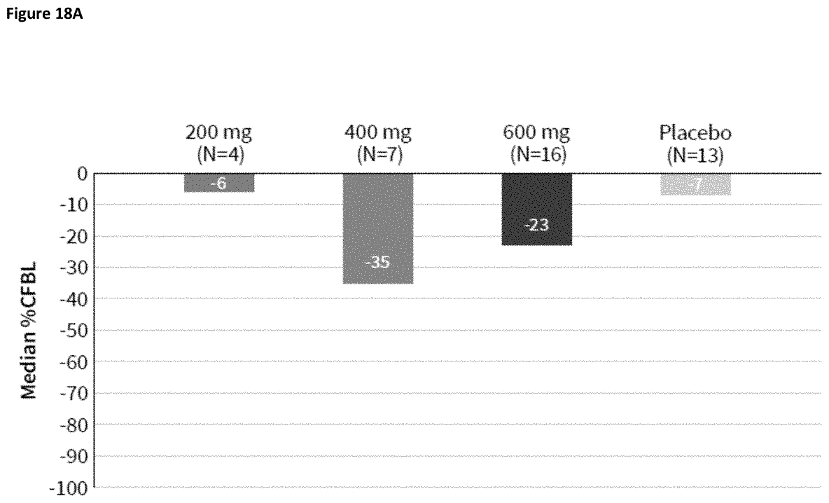

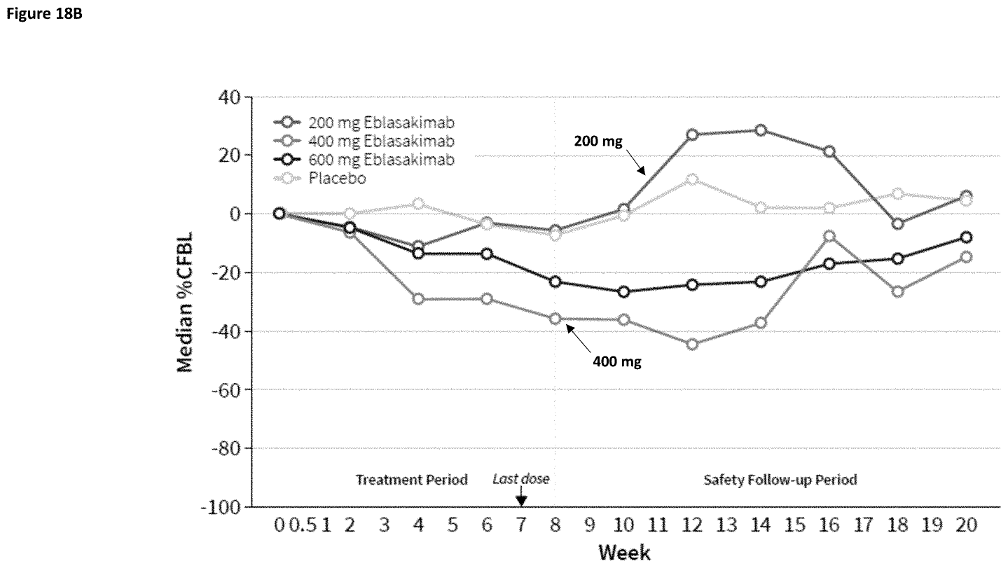

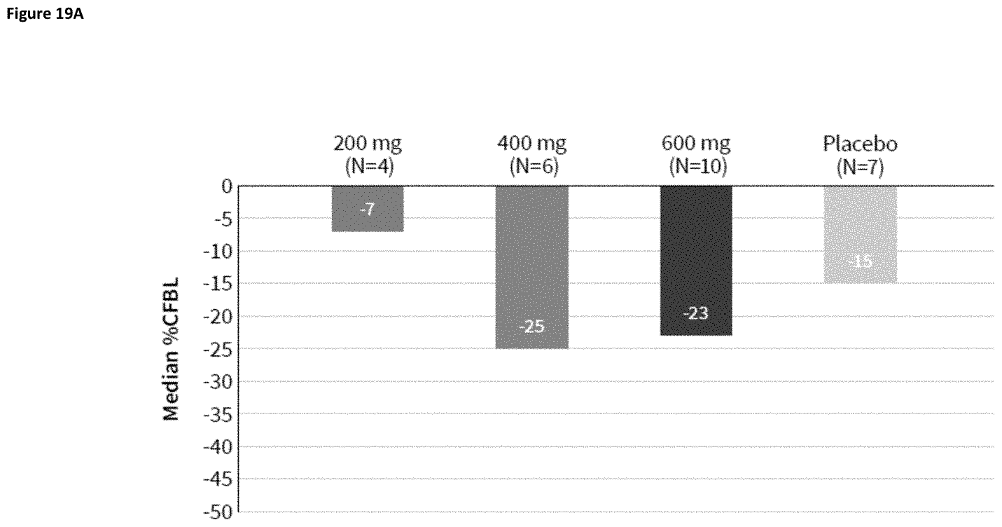

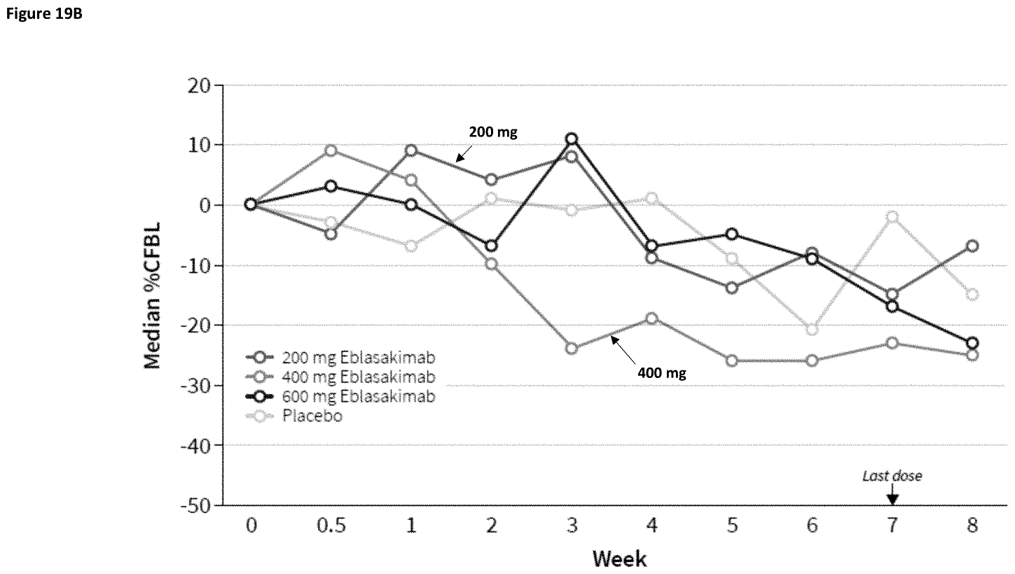

In the mITT population, eblasakimab reduced levels of pharmacodynamic markers IgE, TARC/CCL17, and LDH in the 400 mg and 600 mg dose groups after 8 weeks of once-weekly treatment, with a significant difference between 600 mg vs placebo for TARC/CCL17 (least squares [ls] mean of −62.23 vs −17.83, P<0.001) (FIGS. 17 to 19).

Reductions from baseline were observed as early as the first post-baseline assessment for TARC/CCL17 (day 4), IgE (day 15) and LDH (day 15).

In general, serum biomarkers remained suppressed in the eblasakimab groups for 4-6 weeks following the last dose.

End-of-study values for total IgE and TARC/CCL17 were no different than placebo, a trend also observed for LDH (data not shown).

At the Excluded site, median % changes from baseline in total IgE, TARC/CCL17, and LDH level with 600 mg eblasakimab were substantially less than those of the corresponding mITT 600 mg dose group and similar to placebo at week 8 (P=0.272 vs. placebo for TARC/CCL17; FIG. 17A and data not shown).

### CONCLUSIONS

In this small Phase 1b multiple ascending dose study, eblasakimab, a monoclonal IL-13Rα1 directed antibody, reduced circulating levels of AD-associated pharmacodynamic biomarkers TARC/CCL17, total IgE and LDH.

In this study, biomarker responses were greatest in the 400 mg and 600 mg dose groups and were not further reduced at the higher dose group. Among the biomarkers analyzed, TARC/CCL17 and LDH showed the greatest decrease from baseline levels with eblasakimab treatment. This general suppression of biomarker levels supports the clinical responses and improvements in patient-reported outcomes observed in this trial, as evidenced by reductions in measures of AD severity (see Example 2).

These biomarker results are also consistent with findings in the literature reported for other existing approved AD treatments (see Hamilton, J D, et al. Clin Exp Allergy. 2021; 51(7):915-931) and show the utility of these markers for characterizing reductions in disease severity in a moderate-to-severe AD population.

These data along with the clinical results of the trial support the further investigation of eblasakimab for the treatment of moderate-to-severe AD.

1. A method of treating atopic dermatitis (for example moderate to severe atopic dermatitis, in particular poorly controlled moderate to severe atopic dermatitis) in a highly allergic patient where the baseline IgE levels have been established and are at a level of at least 10,000 KU/L+/−2,000, by administering an antibody, antigen binding fragment thereof, which is an inhibitor of signalling through IL-13Rα1 by binding the said receptor, or a pharmaceutical composition comprising same.

2. A method according to claim 1 wherein the baseline IgE levels are in the range 10,000+/−2,000 to 30,000+/−6,000 KU/L.

3. A method according to claim 1, wherein the treatment reduces the IgE levels by at least 15% from baseline.

4. A method according to claim 3, wherein the treatment reduces the IgE levels by at least 20% from the baseline.

5. A method according to claim 3, wherein the treatment reduces the IgE levels by at least 30% from baseline, for example reduces said levels 30 to 40%, such as 30, 31, 32, 33, 34, 35, 36, 37, 38, 39 or 40% from baseline.

6. A method according to claim 3, wherein the reduction is observed by about day 15.

7. A method according to claim 3, wherein the reduction is observed by about day 29.

8. A method according to claim 3, wherein the reduction is observed by about day 57.

9. A method according to claim 1, wherein said antibody or binding fragment is administered by parenterally, for example in a treatment cycle comprising a dose in the range 200 mg to 600 mg.

10. A method according to claim 1, wherein following the treatment cycle or cycles and disease modification, maintenance therapy is administered, for example the same dose administered less frequently (for example monthly), or a lower dose (such as 200 mg) administered the same frequency or less frequently (such as about two weekly, about three weekly, or about four weekly.

11. A method according to claim 1, wherein said antibody or binding fragment thereof is administered approximately weekly, (in particular a single treatment cycle, especially 8 weeks).

12. A method according to claim 1, wherein said antibody or binding fragment thereof is administered once approximately every two weeks, (in particular a single treatment cycle, especially 8 weeks).

13. A method according to claim 1, wherein said antibody or binding fragment thereof is administered once approximately every three weeks, (in particular a single treatment cycle, especially 8 weeks).

14. A method according to claim 1, wherein the antibody or binding fragment thereof is administered once approximately every four weeks (for example monthly), (in particular a single treatment cycle, especially 8 weeks).

15. A method according to claim 1, wherein a loading dose in the range 400 to 900 mg, for example 400, 500, 600, 700, 800 or 900 mg is employed before administration of the treatment cycle.

16. A method according to claim 1, wherein the treatment does not comprise a loading dose.

17. A method thereof according to claim 1, wherein the dose is 200 mg.

18. A method according to claim 1, wherein the dose is in the range 350 to 450 mg, such as 400 mg.

19. A method according to claim 1, wherein the dose is 600 mg.

20. A method according to claim 1, wherein the treatment cycle comprises, a first dose at 600 mg, followed by three weekly doses of 400 mg, for example wherein the treatment cycle is repeated twice i.e. two treatment cycles lasting 8 weeks, in particular day 1 600 mg, approximately day 8 400 mg, approximately day 15 400 mg, approximately day 22 400 mg, approximately day 29 600 mg, approximately day 36 400 mg, approximately day 43 400 mg, and approximately day 50 400 mg are administered.

21. A method according to claim 1, wherein disease modification, occurs by day 4, wherein day 1 is the first administration of the antibody or binding fragment thereof, for example wherein disease modification is assessed by a reduction in EASI score.


SEQUENCE LISTING (63 sequences)

SEQ ID NO: 1 - protein, 10 aa, synthetic construct
  REGION 1..10: note = CDRH1
GYSFTSYWIG

SEQ ID NO: 2 - protein, 10 aa, synthetic construct
  REGION 1..10: note = CDRH2
VIYPGDSYTR

SEQ ID NO: 3 - protein, 9 aa, synthetic construct
  REGION 1..9: note = CDRH3
  SITE 1: note = MISC_FEATURE - Xaa denotes Phe, Met, Gln, Leu or Val
  SITE 6: note = MISC_FEATURE - Xaa denotes Ser or Ala
  SITE 7: note = MISC_FEATURE - Xaa denotes Phe, Leu, Ala or Met
  SITE 9: note = MISC_FEATURE - Xaa denotes Tyr, Gln, Lys, Arg, Trp, His, Ala, Thr, Ser, Asn orGly
XPNWGXXDX

SEQ ID NO: 4 - protein, 9 aa, synthetic construct
  REGION 1..9: note = CDR H3 SEQUENCE
FPNWGALDQ

SEQ ID NO: 5 - protein, 9 aa, synthetic construct
  REGION 1..9: note = CDR H3 SEQUENCE
VPNMGSLDT

SEQ ID NO: 6 - protein, 9 aa, synthetic construct
  REGION 1..9: note = CDR H3 SEQUENCE
FPNWGSMDA

SEQ ID NO: 7 - protein, 9 aa, synthetic construct
  REGION 1..9: note = CDR H3 SEQUENCE
FPNWGSLDH

SEQ ID NO: 8 - protein, 9 aa, synthetic construct
  REGION 1..9: note = CDR H3 SEQUENCE
MPNWGSFDY

SEQ ID NO: 9 - protein, 9 aa, synthetic construct
  REGION 1..9: note = CDR H3 SEQUENCE
MPNWGSFDT

SEQ ID NO: 10 - protein, 9 aa, synthetic construct
  REGION 1..9: note = CDR H3 SEQUENCE
MPNWGSLDH

SEQ ID NO: 11 - protein, 9 aa, synthetic construct
  REGION 1..9: note = CDR H3 SEQUENCE
MPNWGSFDS

SEQ ID NO: 12 - protein, 9 aa, synthetic construct
  REGION 1..9: note = CDR H3 SEQUENCE
MPNWGSLDT

SEQ ID NO: 13 - protein, 9 aa, synthetic construct
  REGION 1..9: note = CDR H3 SEQUENCE
MPNWGSLDA

SEQ ID NO: 14 - protein, 9 aa, synthetic construct
  REGION 1..9: note = CDR H3 SEQUENCE
MPNWGSLDN

SEQ ID NO: 15 - protein, 9 aa, synthetic construct
  REGION 1..9: note = CDR H3 SEQUENCE
MPNWGALDS

SEQ ID NO: 16 - protein, 9 aa, synthetic construct
  REGION 1..9: note = CDR H3 SEQUENCE
MPNWGSLDN

SEQ ID NO: 17 - protein, 9 aa, synthetic construct
  REGION 1..9: note = CDR H3 SEQUENCE
MPNWGSLDY

SEQ ID NO: 18 - protein, 9 aa, synthetic construct
  REGION 1..9: note = CDR H3 SEQUENCE
MPNWGSFDH

SEQ ID NO: 19 - protein, 9 aa, synthetic construct
  REGION 1..9: note = CDR H3 SEQUENCE
MPNWGSLDS

SEQ ID NO: 20 - protein, 9 aa, synthetic construct
  REGION 1..9: note = CDR H3 SEQUENCE
MPNWGSLDG

SEQ ID NO: 21 - protein, 9 aa, synthetic construct
  REGION 1..9: note = CDR H3 SEQUENCE
VPNWGSLDG

SEQ ID NO: 22 - protein, 28 aa, synthetic construct
  REGION 1..28: note = CDR H3 SEQUENCE
CARFPNWGSLDHWGQGTLVTVSSASIKG

SEQ ID NO: 23 - protein, 28 aa, synthetic construct
  REGION 1..28: note = CDR H3 SEQUENCE
CARMPNWGSLDHWGQGTLVTVSSASTKG

SEQ ID NO: 24 - protein, 28 aa, synthetic construct
  REGION 1..28: note = CDR H3 SEQUENCE
CARMPNWGSFDYWGQGTLVTVSSASIKG

SEQ ID NO: 25 - protein, 12 aa, synthetic construct
  REGION 1..12: note = CDR H3 SEQUENCE
VRMPNWGSLDHW

SEQ ID NO: 26 - protein, 27 aa, synthetic construct
  REGION 1..27: note = CDR H3 SEQUENCE
VRMPNWGSLDHWGQGTLVTVSSADIKG

SEQ ID NO: 27 - protein, 27 aa, synthetic construct
  REGION 1..27: note = CDR H3 SEQUENCE
ARMPNWGSLDHWGQGTLVTVSSASIKG

SEQ ID NO: 28 - protein, 25 aa, synthetic construct
  REGION 1..25: note = CDR H3 SEQUENCE
FPNWGSFDYWGQGTLVTVSSASIKG

SEQ ID NO: 29 - protein, 9 aa, synthetic construct
  REGION 1..9: note = CDR H3 SEQUENCE
VPNWGSLDA

SEQ ID NO: 30 - protein, 9 aa, synthetic construct
  REGION 1..9: note = CDR H3 SEQUENCE
FPNWGSFDY

SEQ ID NO: 31 - protein, 12 aa, synthetic construct
  REGION 1..12: note = CDRL1
RASQSISSSYLA

SEQ ID NO: 32 - protein, 7 aa, synthetic construct
  REGION 1..7: note = CDRL2
GASSRAT

SEQ ID NO: 33 - skipped

SEQ ID NO: 34 - protein, 5 aa, synthetic construct
  REGION 1..5: note = CDRL3 SEQUENCE
QRYAT

SEQ ID NO: 35 - protein, 5 aa, synthetic construct
  REGION 1..5: note = CDRL3 SEQUENCE
QRYST

SEQ ID NO: 36 - protein, 5 aa, synthetic construct
  REGION 1..5: note = CDRL3 SEQUENCE
QMYST

SEQ ID NO: 37 - protein, 5 aa, synthetic construct
  REGION 1..5: note = CDRL3 SEQUENCE
QQVGT

SEQ ID NO: 38 - protein, 5 aa, synthetic construct
  REGION 1..5: note = CDRL3 SEQUENCE
QQVST

SEQ ID NO: 39 - protein, 5 aa, synthetic construct
  REGION 1..5: note = CDRL3 SEQUENCE
QQYST

SEQ ID NO: 40 - protein, 5 aa, synthetic construct
  REGION 1..5: note = CDRL3 SEQUENCE
QSYST

SEQ ID NO: 41 - protein, 5 aa, synthetic construct
  REGION 1..5: note = CDRL3 SEQUENCE
QQYAT

SEQ ID NO: 42 - protein, 5 aa, synthetic construct
  REGION 1..5: note = CDRL3 SEQUENCE
QQYSS

SEQ ID NO: 43 - protein, 5 aa, synthetic construct
  REGION 1..5: note = CDRL3 SEQUENCE
QTYST

SEQ ID NO: 44 - protein, 5 aa, synthetic construct
  REGION 1..5: note = CDRL3 SEQUENCE
QQYGS

SEQ ID NO: 45 - protein, 5 aa, synthetic construct
  REGION 1..5: note = CDRL3 SEQUENCE
QQYAS

SEQ ID NO: 46 - protein, 5 aa, synthetic construct
  REGION 1..5: note = CDRL3 SEQUENCE
QQYEA

SEQ ID NO: 47 - protein, 5 aa, synthetic construct
  REGION 1..5: note = CDRL3 SEQUENCE
QQYET

SEQ ID NO: 48 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARFPNWGSFDYWGQGTLVTVSS

SEQ ID NO: 49 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSS

SEQ ID NO: 50 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSS

SEQ ID NO: 51 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSS

SEQ ID NO: 52 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEI

SEQ ID NO: 53 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEI

SEQ ID NO: 54 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEI

SEQ ID NO: 55 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 443 aa, synthetic construct
  REGION 1..443: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 61 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC